SEQUENCE LISTING (2 sequences)

SEQ ID NO: 1 - protein, 482 aa, synthetic construct
MLGRKIAGTCLRANVPSVAATSSTPATQTRNSHTIWYPDAKFERQFKTGGTLGKLWMSERVSDFDEKIGLDKLEKLAYSDPVMSDNYSGKQREKNLENMILNFGPQHPAAHGVLRLVLKLEGEVIIKAIPHIGLLHRATEKLIEHKTYTQALPYFDRLDYVSMMCNEQAWSLAVEKLLGIDIPTRAKYIRTLMGELTRIQNHIMGITTHALDVGAMTPFFWMFEEREKLFEFSERVSGARMHANYVRPGGVAWDLPIGLMDDIYDWAIKFPERIDELEDMLTENRIWKARTIDIGLVSAADALNWGFSGVMVRGSGIKQDVRKTEPYDAYADMEFDVPIGTKGDCYDRYLCRIEEMRQSLNIVHQCLNKMPAGEIKVDDHKVVPPKRAEMKENMESLIHHFKFFTEGFQVPPGATYVPIEAPKGEFGVYLVADGTGKPYRCFIRAPGFAHLAAIHDVCYMSLIADIVAVIGTMDIVFGEVDR

SEQ ID NO: 2 - protein, 464 aa, synthetic construct
MAATMLRSLSRLGRPSVLLNSNVSSPACALIQTRQKQWQPDVEWVEQFSGVVMYPSPSVAKWTPPPWNDKDPPAQKDLSNLTINFGPQHPAAHGVLRLVMELSGESVKKCDPHIGLLHRGTEKLIEYKTYLQALPYFDRLDYVSMMCNEQAYSLAVEKLLNIQAPPRAQWIRVLFGEMTRIMNHIMGITTHALDIGAMTPFFWMFEEREKLFEFYERVSGARMHAAYVRPGGVHQDMPLGLMDDIYEWCKNFSIRIDEVEEMLTNNRIWKNRTVGIGVIGAEEALNYGFSGVMLRGSGIKWDLRKSQPYDKYDEVEFDMAIGTNGDCYDRYLCRVEEMRQSLRIMHQCLNKMPPGEIKVDDAKIAPPKRSEMKTSMESLIHHFKLYTEGYQVPPGATYTAIEAPKGEFGVYLVSDGSSRPYRCKIKAPGFAHLAGLDRMSQGHMLADVVAIIGTQDIVFGEVDR

(19) United States
(12) Patent Application Publication
(10) Pub. No.: US 2025/0017895 A1
(43) Pub. Date: Jan. 16, 2025

(54) FLAVANOID CONTAINING COMPOSITIONS AND METHODS OF USE THEREOF FOR THE TREATMENT OF MITOCHONDRIAL DISORDERS

(71) Applicant: THE CHILDREN'S HOSPITAL OF PHILADELPHIA, Philadelphia, PA (US)

(72) Inventors: Marni J. FALK, Voorhees, NJ (US); Neal D. MATHEW, Philadelphia, PA (US); Eiko NAKAMARU-OGISO REINGEWIRTZ, Havertown, PA (US); Min PENG, Sewell, NJ (US); Erzsebet POLYAK, Madison, WI (US); Bhumi SHAH, Dayton, NJ (US)

(73) Assignee: THE CHILDREN'S HOSPITAL OF PHILADELPHIA, Philadelphia, PA (US)

(21) Appl. No.: 18/713,342

(22) PCT Filed: Nov. 29, 2022
(86) PCT No.: PCT/US2022/080566

§ 371 Date: May 24, 2024

Related U.S. Application Data
(60) Provisional application No. 63/284,003, filed on Nov. 29, 2021.

(51) Int. Cl. A61K 31/353; A61K 31/095; A61K 31/197; A61K 31/455; A61K 31/57; A61K 31/7004; A61P 43/00

(52) U.S. Cl. CPC A61K 31/353; A61K 31/095; A61K 31/197; A61K 31/455; A61K 31/57; A61K 31/7004; A61P 43/00

(57) ABSTRACT

This disclosure is directed to methods of treating or inhibiting mitochondrial dysfunction or mitochondrial disease in a subject comprising administration of and effective amount of one or more of (+) Epicatechin (−) epicatechin, 11-hydroxyprogesterone, and 11-β-hydroxypregnenolone and other efficacious agents in a pharmaceutically acceptable carrier. Agents can be administered alone or in certain synergistic combinations. The compositions and methods described effectively reduce or alleviate primary respiratory chain dysfunction symptoms, and have efficacy for improving cellular resiliency, stress resistance, and symptoms associated with primary, secondary, or acute respiratory chain dysfunction.

### CROSS-REFERENCE TO RELATED APPLICATION

This application claims priority to U.S. Provisional Patent Application No. 63/284,003 filed on Nov. 29, 2021 which is incorporated herein by reference in its entirety.

### GRANT STATEMENT

This invention was made with government support under grant number R01-HD065858 awarded by the National Institutes of Health. The US government has certain rights in the invention.

### INCORPORATION-BY-REFERENCE OF MATERIAL SUBMITTED IN ELECTRONIC FORM

The contents of the electronic sequence listing (CHOP-135PCT.xml; Size: 4,096 bytes; and Date of Creation: Nov. 29, 2022) is herein incorporated by reference in its entirety.

### TECHNICAL FIELD

This invention relates to the fields of mitochondrial disease and aberrant respiratory chain function. More specifically, the invention provides flavonoid compositions and methods of use thereof having efficacy for improving cellular resiliency, stress resistance, and symptoms associated with primary, secondary, or acute respiratory chain dysfunction.

### BACKGROUND OF THE INVENTION

Several publications and patent documents are cited throughout the specification in order to describe the state of the art to which this invention pertains. Each of these citations is incorporated herein by reference as though set forth in full.

Primary mitochondrial respiratory chain (RC) disease afflicts at least 1 in 4,300 people with multi-system manifestations for which there currently are no proven effective treatment other than empirically prescribed antioxidants and cofactors. Complex I deficiency is the most frequently encountered single mitochondrial respiratory chain enzyme deficiency in patients with a mitochondrial disorder. Although specific genotype-phenotype correlations are very difficult to identify due to extensive pleiotropy, locus heterogeneity, and allelic heterogeneity, the majority of patients present with neurologic or muscular symptoms such as metabolic stroke, leukodystrophy, peripheral neuropathy, autonomic dysfunction, fatigue, exercise intolerance, myopathy, cardiomyopathy, arrhythmia, liver or kidney disease, vision loss, and hearing loss. The average mitochondrial disease patient suffers up to 16 symptoms, which can be highly variable in onset and severity, but are often induced or exacerbated by stressors that can lead to severe morbidity or death. The poor genotype-phenotype correlations can make establishing a diagnosis a challenge. The classical way to establish a respiratory chain complex(es) deficiency in patients is by performing polarographic and/or spectrophotometric measurements of the enzyme in a muscle biopsy or other patient-derived material (liver or heart biopsy, cultured skin fibroblasts). Complexes I, III, IV, and V subunits are encoded by both mitochondrial DNA (mtDNA) and nuclear DNA, while complex II subunits are encoded only by nuclear DNA. Pathogenic mutations have been identified in many different structural subunits of the respiratory chain, respiratory chain assembly factors, mtDNA-encoded transfer or ribosomal RNAs, and a host of nuclear genes effecting nucleotide metabolism, mitochondrial DNA replication and repair, oxidative stress, and mitochondrial dynamics such as fission and fusion. In recent years, the increasing possibilities for diagnostic molecular genetic tests of large gene panels, exomes, and even entire genomes has led to the identification of many novel genetic defects causing respiratory chain disease, with more than 350 genes now known to play a causal role in every possible Mendelian or maternal inheritance pattern.

Respiratory chain complex disorders result in reduced enzyme activity, impaired mitochondrial membrane potential and oxygen consumption capacity, altered mitochondrial morphology and/or cellular mitochondrial amount, impaired energy generation in the form of adenosine triphosphate (ATP), altered redox balance of nicotinamide dinucleotide (NADH, NAD+) metabolism, and also induce secondary effects at the cellular level, globally disrupting signaling pathways. Pathways particularly affected involve nutrient-sensing signaling networks, aberrant autophagy and mitophagy, increased cytosolic translation, increased lysosomal numbers, and globally elevated reactive oxygen species production. Also common is glutathione depletion, along with a wide range of secondary intermediary metabolic alterations, stressor sensitivity, oxidative stress, proteotoxic stasis and stress, and cell death.

Catechins and epicatechins are phytochemical compounds found in high concentrations in a variety of plant-based foods and beverages. Based on their structure, these compounds are classified as flavanols and include the following compounds: catechin, epicatechin, epigallocatechin, epicatechin gallate, and epigallocatechin gallate. High concentrations of catechin can be found in red wine, broad beans, black grapes, apricots and strawberries. Epicatechin concentrations are high in apples, blackberries, broad beans, cherries, black grapes, pears, raspberries, and cocoa/chocolate. A racemic mixture of (+) and (−) epicatechin is naturally found in most of these foods. (−) epicatechin is generally available as a generic health supplement. Finally, epigallocatechin, epicatechin gallate, and epigallocatechin gallate are found in high concentrations in both black and green tea. There have been reports in the literature that certain flavonoids may have mitochondrial protective properties.

Currently, very few safe and effective therapies, and no cures, for mitochondrial respiratory chain diseases have been described. Clearly, an urgent need exists for new therapeutic approaches for the amelioration of the symptoms of RC disease.

### SUMMARY OF THE INVENTION

In accordance with the present invention, compositions and methods for the prevention and treatment of mitochondrial disease are provided. In one embodiment, a composition comprising effective amounts of (+) epicatechin in a pharmaceutically acceptable carrier for preventing or alleviating symptoms of mitochondrial disease are disclosed. In certain embodiments, the composition can further comprise one or more of (−) epicatechin, 11-β-hydroxypregnenolone, 11-hydroxyprogesterone, probucol, glucose, N-acetylcysteine, cysteamine bitartrate, and nicotinic acid, niacin, or nicotinamide, administered separately or in combination. In other embodiments, effective amounts of one or more of (+) epicatechin, (−) epicatechin, 11-β-hydroxypregnenolone, 11-hydroxyprogesterone, probucol, glucose, N-acetylcysteine, cysteamine bitartrate, and nicotinic acid, niacin, or nicotinamide containing compositions are administered separately or in combination. In certain embodiments, these agents act additively. In other embodiments, these agents act synergistically. Also provided is a method for alleviating symptoms associated with mitochondrial disease, comprising administration of the compositions described above to a patient in need thereof. Symptoms to be alleviated include, without limitation, one or more of muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, dyslipidemia, cardiomyopathy, arrhythmia, anemia, failure to thrive, over or underweight, developmental delay, neurodevelopmental regression, cognitive decline and memory impairment, Parkinsonism, dystonia, liver dysfunction or failure, infertility, metabolic instability, stressor-induced acute decompensation, DLD disease, mitophagy disorders, mitochondrial lipid biogenesis disorders, mitochondrial cofactor disorders, and secondary mitochondrial disorders including but not limited to resulting from toxins, drugs, age, prescribed or illicit medications, smoking, alcohol, environmental exposures, obesity, and genetic disorders that secondarily impair mitochondrial function, structure, or activities. In certain embodiments, the mitochondrial disease is selected from the group consisting of Complex I disease, Complex II disease, Complex III disease, Complex IV disease, Complex V disease, multiple respiratory chain complex disease, adenine nucleotide translocase deficiency, pyruvate dehydrogenase deficiency, mitochondrial depletion disease, multiple mitochondrial DNA deletions disease, mitochondrial DNA maintenance defects, mitochondrial translation defects, mitochondrial nucleotide import disease, Friedreich's ataxia, Leber's Hereditary Optic Neuropathy, Kearns-Sayre Syndrome, Pearson Syndrome, Mitochondrial Myopathy, Mitochondrial Encephalomyopathy with Lactic Acidosis and Stroke-Like Episodes syndrome, Myoclonic Epilepsy and Ragged Red Fibers syndrome, Neurogenic Ataxia and Retinitis Pigmentosa, Mitochondrial Neuro-Gastrointestinal Encephalomopathy, maternally-inherited diabetes and deafness, FBXL4 mitochondrial encephalomyopathy, primary lactic acidosis, Leigh syndrome, Leigh syndrome spectrum, Leigh-like syndrome, and multi-system mitochondrial disease. In other embodiments, the disease in Complex I mitochondrial disease.

In certain aspects the methods comprise administrations of compositions comprising synergistic amounts of (+) epicatechin plus (−) epicatechin or (+) epicatechin plus 11-β-hydroxypregnenolone or (−) epicatechin and 11-β-hydroxypregnenolone.

### BRIEF DESCRIPTION OF THE DRAWINGS

FIG. 1: Proposed mechanism of action of the two isoforms. (−) Epicatechin and (+) Epicatechin act through mimicry of the endogenous mitochondrial steroid 11-β-hydroxypregnenolone, a potent mitochondrial steroid that induces mitochondrial biogenesis.

FIGS. 2A-2E: Nematode lifespan. gas-1(fc21) worms that have an autosomal recessive missense mutation in the mitochondrial complex Indufs2^{-/-} subunit have short lifespan at 20° C. relative to wild-type (N2 Bristol) worms (when analysis is performed without use of FUDR) that was significantly rescued with 10 nM (+) epicatechin, as well as 1 nM and 100 nM (+) epicatechin to a significant but somewhat lesser degree. The rescue of gas-1(fc21) short lifespan by a 4-log dose range of (+) epicatechin is also shown in FIG. 2 (no FUDR used), which shows that relative to N2 Bristol wild-type worms (black line), median and maximal lifespans of short-lived gas-1(fc21) mutant worms (red line) are significantly improved with (+) epicatechin, where the greatest effect was seen with 10 nM treatment (green line). FIG. 2C, the Table below FIGS. 2A and 2B indicates specific values and P values calculated in Graphpad Prism v5.0 for graph shown in FIG. 2B (survival data is shown in days). FIGS. 2D-2F: Comparative analysis of 4-log concentration range of (+) epicatechin (Epi plus), (−) epicatechin (Epi minus), and 11-β-hydroxypregnenolone (11OHP) on lifespan of gas-1(fc21) ndufs2^{-/-} complex I disease mutant worms. FUDR was used in this series of experiments to allow for concurrent analysis of multiple conditions by manual lifespan assay that requires counting of all animals daily with manual prodding, as FUDR prevents offspring of animals under study from growing up to complicate the parental stage lifespan analysis—this is the origin of the relative prolongation of gas-1(fc21) lifespan relative to N2 control as gas-1(fc21) worms are known to have lifespan extension with FUDR whereas N2 worms do not. Table 2G provides a detailed summary of the data shown, with p values demonstrating statistically significant differences of treatment group relative to buffer-only control, and median, mean, and maximal worm survival values shown in days. Significant lifespan rescue of gas-1(fc21) ndufs2^{-/-} complex I disease mutant worms compared to buffer-only treated gas-1(fc21) worms was seen with 1 nMol and 10 nMol (+) epicatechin, 10 nMol and 100 nMol (−) epicatechin, and 1 nMol and 10 nMol 11-β-hydroxypregnenolone. The greatest recue among these three drugs studied was seen in median (23 days with 1 nMol) and maximum (31 days with 10 nMol) survival with (+) epicatechin as compared to buffer-only gas-1(fc21) worms that had a median survival of 19 days and maximum survival of 27 days.

FIGS. 3A-3B: (+) Epicatechin (10 nM) treatment for 24 hours improves C. elegans nutrient sensing signaling network (NSSN) gene expression alterations in gas-1(fc21) mitochondrial complex I ndufs2^{-/-} mutant adult worms relative to wild-type (N2 Bristol) control worms (DMSO buffer control). SOD2 (manganese superoxide dismutase, mnSOD) is the major endogenous antioxidant scavenging system that was upregulated in gas-1(fc21) worms and normalized with 24-hour (+) epicatechin treatment to that of wild-type levels. Daf-16 is the FOXO orthologue in worms that regulates longevity and stress responses, that was downregulated in gas-1(fc21) mutant worms but substantially upregulated with (+) epicatechin treatment. Par-4 is the LKB1 (STK11) orthologue in worms that functions as a serine threonine kinase involved in cell division and fate decisions that was downregulated in gas-1(fc21) and increased with 24 hour (+) epicatechin treatment. RQ, relative quantitation. FIG. 3C shows the signaling effects of (+) epicatechin and 11-β-hydroxypregnenolone treatment in gas-1(fc21) worms relative to DMSO-only buffer exposed gas-1(fc21) worms or DMSO-only buffer exposed wild-type (N2) worms for 24 hours on RNAseq transcriptome profiling expression data aggregated as KEGG biochemical and signaling pathways. Of note, MAPK signaling pathway expression was uniquely and substantially modulated by both flavonoid treatments. Pathways marked with a star are also positively modulated in this same mitochondrial disease animal model by combination glucose+nicotinic acid+N-acetylcysteine triple therapy.

FIGS. 4A-4E: (+) Epicatechin improves multiple aspects of mitochondrial pathophysiology in mitochondrial respiratory chain (RC) deficient C. elegans gas-1(fc21) ndufs2^{-/-} mutant worms. FIG. 4A: Mitochondrial oxidant burden at 24 hours, which was significantly increased in gas-1(fc21) ndufs2^{-/-} worms relative to wild-type worms but significantly rescued with 1 nMol, 10 nMol, and 100 nMol (+) epicatechin. Data shows results from 2 biological replicate independent experiments. ***, p<0.001. FIG. 4B: Mitochondrial content at 24 hours, which was significantly reduced in gas-1(fc21) ndufs2-worms relative to wild-type worms but significantly rescued with 1 nMol, 10 nMol, and 100 nMol (+) epicatechin. Data shows results from 2-3 biological replicate independent experiments. ***, p<0.001. FIG. 4C: Mitochondrial membrane potential at 24 hours, which was significantly reduced in gas-1(fc21) ndufs2^{-/-} worms relative to wild-type worms but significantly reduced in gas-1(fc21) ndufs2^{-/-} worms relative to wild-type worms but significantly recued with 10 nMol (+) epicatechin. Data shows results from 2 biological replicate independent experiments. ***, p<0.001. FIG. 4D: Mitochondrial oxidant burden at 72 hours, which was significantly increased in gas-1(fc21) ndufs2^{-/-} worms relative to wild-type worms but significantly rescued with 10 nMol (+) epicatechin. Data shows results from 2 biological replicate independent experiments. ***, p<0.001. FIG. 4E Mitochondrial content at 72 hours, which was significantly reduced in gas-1(fc21) ndufs2^{-/-} worms relative to wild-type worms but significantly rescued with 10 nMol (+) epicatechin. Data shows results from 2 biological replicate independent experiments. ***, p<0.001. In each panel, n conveys the number of animals studied per conditions. Bars and error bars convey mean and standard error, respectively.

FIGS. 5A and 5B: Worm neuromuscular activity as assayed by body bend rate analysis was reduced in gas-1(fc21) mitochondrial complex I disease ndufs2^{-/-} worms relative to wild-type (N2 Bristol) worms. (+) Epicatechin, (−) epicatechin, and 11-β-hydroxypregnenolone (“Pre”) at 100 nMol each significantly rescued the impaired whole animal neuromuscular function of gas-1(fc21) mitochondrial complex I disease ndufs2-mutant worms toward that of wild-type (N2 Bristol) worms. **, p<0.01. ***, p<0.001.

FIGS. 6A-6D: (+) Epicatechin modulates human cell viability and mitigates cell death in pharmacologic or genetic RC disease. FIG. 6A: Human podocytes (renal glomerular cells) exposed to low-dose (12.5 nM) of a mitochondrial complex I inhibitor (rotenone) had 45% cell death that was improved when treated with 10 nM or 100 nM (+) epicatechin but not with 1 nM (+) epicatechin is shown in FIG. 6B. FIG. 6C shows transmitochondrial cybrid cell line derived from a human fibroblast cell line with a pathogenic mtDNA variant, m586G>A had significantly improved cell survival (*p<0.01) when treated for 72 hours in galactose media (that stresses cells by requiring aerobic mitochondrial metabolism to generate chemical energy in the form of adenosine triphosphate (ATP) rather than anaerobic glycolysis). Bars convey mean and standard error from 3 biological replicate independent experiments. FIG. 6D: FBXL4-human disease patient fibroblasts, representing a nuclear-encoded genetic disease that causes mitochondrial respiratory chain deficiency, mitochondrial depletion, primary lactic acidosis and Leigh syndrome. FBXL4-fibroblasts had increased cell death when grown for 72 hours in galactose, which was significantly rescued by 20 nM (+) epicatechin. Bars convey mean and standard error from 3 biological replicate independent experiments. * p<0.05.

FIGS. 7A-7B: (+) Epicatechin at 1 nMol or 10 nMol concentrations for 24 hours improved mitochondrial content by 70% in human fibroblasts from a patient with FBXL4/disease (which is a known mitochondrial depletion disorder causing multi-system symptoms), as measured by fluorescence activated cell sorting (FACS) analysis of Mitotracker Green fluorescence in galactose media. Nutritional status (galactose) can potentiate (+) epicatechin effects. **, p<0.01. n=3 biological replicate independent experiments in FIG. 7A, and a single representative biological replicate comparative experiment in FIG. 7B.

FIG. 8: CRISPR/Cas9-generated ndufs2^{-/-} zebrafish. C. elegans gas-1(fc21) worms have a homozygous Arg→Lys mutation (p.R290K) in the human mitochondrial respiratory chain complex I NDUFS2 subunit orthologue. A Danio rerio (D. rerio, zebrafish) ndufs2^{-/-} strain generated at CHOP with CRISPR/Cas9 technology (SEQ ID NO: 2) has a homozygous 16 base pair deletion that causes a frameshift mutation and premature stop codon. Both mutant strains are animal models for NDUFS2-based complex I autosomal recessive human disease. Blue shading highlights the sight of the resulting ndufs2 protein mutation effects in both species. Worm sequence is SEQ ID NO: 1. Zebrafish sequence is SEQ ID NO: 2.

FIG. 9: ndufs2-zebrafish phenotypes are shown in zebrafish larvae at 5 day post fertilization (dpf). Blue arrow highlights gray liver indicative of liver disease that occurs in the ndufs2^{-/-} zebrafish. ndufs2^{-/-} mutant fish are also smaller in size indicative of developmental delay, do not inflate their swim bladders, have abnormal muscle tone, and die early by 9-10 dpf.

FIG. 10: ndufs2^{-/-} zebrafish have selectively and specifically reduced respiratory chain complex I enzyme activity relative to homozygous and heterozygous wild-type controls, with normal activities of complexes II and IV as well as citrate synthase (CS), which is a marker of mitochondrial content.

FIG. 11: ndufs2^{-/-} zebrafish larvae display stressor hypersensitivity upon further complex I inhibition given their chronic severe complex I deficiency (as shown in FIG. 10), with reduced dark-induced swimming activity (blue shading=0% light; cream shading=60% light) upon exposure to low-dose complex I inhibition with 12 nMol rotenone for 4 hours at 7 days post fertilization (dpf). Each line conveys compiled results from all wells studied per condition in a single experiment, with 1 zebrafish larvae per well in a 96-well plate.

FIG. 12: (+) Epicatechin significantly rescued reduced neuromuscular function as evidenced by impaired swimming activity of ndufs2^{-/-} zebrafish exposed to low-dose (12 nMol) rotenone. Swimming activity (Zebrabox, Viewpoint) was quantified in the first five-minute periods of three consecutive dark cycles across 3 independent biological replicate experiments. Activity score was normalized to percent of wild-type concurrent sibling controls. 100 nMol of (+) epicatechin pre-treatment from 5 dpf significantly improved dark-induced swimming impairment that was caused by subsequent acute low-dose rotenone exposure at 7 dpf, with increase in the mutant zebrafish's swimming activity by ˜25% toward that of wild-type controls. Each symbol indicates one fish. *, p<0.05. ***, p<0.001.

FIG. 13: 11-β-hydroxypregnenolone significantly rescued reduced neuromuscular function as evidenced by impaired swimming activity of AB (wild-type) zebrafish exposed to high-dose (70 uMol) rotenone. AB zebrafish had significantly reduced swimming activity upon exposure to high-dose (70 nM) rotenone (complex I inhibitor) exposure for 4 hours on 7 dpf. Pre-treatment of the AB zebrafish with 2 nM of 11-β-hydroxypregnenolone from 5 dpf significantly reduced dark-induced swimming impairment upon subsequent acute high-dose rotenone exposure of AB larvae at 7 dpf, with increase in the pretreated AB zebrafish's swimming activity by ˜25% toward that of AB controls. Each symbol indicates one fish. **, p<0.01. ***, p<0.001.

FIGS. 14A-14B. In vitro (+) epicatechin and 11-β-hydroxypregnenolone treatments in human kidney podocyte cells maintains viability with direct mitochondrial respiratory chain (RC) pharmacologic inhibition and improves mitochondrial content in genetic RC disease. FIG. 14A and FIG. 14B: Human podocyte viability upon RC inhibition was significantly improved with 10 nM (+) epicatechin (red bars/black circle) in either glucose (panel A) or galactose (panel B) media, where the latter requires mitochondrial OXPHOS capacity to generate ATP. Glucose media: 10% FBS, RPMI, 11 mM glucose; 35 nM rotenone (ROT)±10 nM (+) epicatechin (‘EPI(+)’), 1 nM 11-hydroxyprogesterone (11-OHP, green), or 10 nM (−) epicatechin (‘EPI(−)’, blue) for 48 hours. Data are shown as mean±SEM of 3 independent trials. (+) epicatechin was more effective than (−) epicatechin. Galactose media. 10% FBS, DMEM-no glucose, 10 mM galactose; 25 nM rot (black bar)+10 nM EPI(+), 1 nM 11-OHP (green), or 10 nM or 100 nM EPI(−) (blue) for 48 hours.

FIGS. 15A-15B: (+) Epicatechin treatment (5 to 10 μg/ml fed in drinking water) of Pdss2^{kd/kd} homozygous missense mutant mice, which have neuromuscular dysfunction, Parkinsonism, and renal glomerular disease due to impaired Coenzyme Q biosynthesis, normalized their reduced complex I-dependent and complex I+II integrated respiratory chain oxidative phosphorylation capacity as compared to wild-type (B6) controls. Pdss2^{kd/kd} mice were already grossly ill with renal disease as evidenced by frank albuminuria at the time (+) Epicatechin treatment was begun at 90-115 days of life, with daily refreshing of their treatment for two months prior to sacrifice and tissue analysis. Polarography was performed by Oxygraph 2K (Oroboros Instruments) in freshly isolated permeabilized skeletal muscle to evaluate mitochondrial complex I (malate+ADP) integrated oxphos capacity (FIG. 15A) and integrated mitochondrial complexes I+II (malate+succinate+ADP) oxphos capacity (FIG. 15B). N conveys number of animals studied per condition, split to display sex-related effects.

FIGS. 16A-16B: (FIG. 16A) (+) Epicatechin treatment significantly increased the mitochondrial content of Pass2^{kd/kd} homozygous missense mutant mice, as quantified in a subset of the same animals described in FIG. 15 by quantifying VDAC (porin) protein expression as a outer mitochondrial membrane marker indicative of mitochondrial amount present in the liver. Significant increase in VDAC expression as quantified by image J analysis of western immunoblot in (+) epicatechin-fed Pdss2^{kd/kd} mice relative to untreated mutant controls (*, p<0.05). (FIG. 16B) Western immunoblot analysis depicting porin expression levels in mouse liver of Pdss2^{kd/kd} mice (1 mouse/lane). F, female. M, male. (+) Epicatechin dosing in drinking water is shown above blot, as 0 (−), 5 μg/mL (5), or 10 μg/mL (10).

FIGS. 17A-17E: Biochemical analysis of mitochondrial amount (citrate synthase activity) and NADH:NAD+ redox balance in coenzyme Q deficient Pdss2^{kd/kd} mice. FIGS. 17A-17B: Citrate synthase activity was spectrophotometrically quantified in isolated mouse liver, and normalized either to mg liver protein (FIG. 17A) or to grams of wet weight tissue (FIG. 17B), which both similarly showed a reduction in citrate synthase activity in Pdss2^{kd/kd} mice relative to B6 controls that was substantially increased in both sexes individually and combined in Pdss2^{kd/kd} mice treated with 5 μg/mL or 10 μg/mL (+) epicatechin, per experimental treatment details shown in FIGS. 15-16 above. These data demonstrate that reduction in mitochondrial content (i.e., mitochondrial depletion) occurs in Pdss2^{kd/kd} mouse liver, and is largely normalized by (+) epicatechin treatment. FIGS. 17C-17D: NAD+ (FIG. 17C), NADH (FIG. 17D), and NADH:NAD+ redox ratio (FIG. 17E) analyses in liver from Pdss2^{kd/kd} mice that have a mitochondrial coenzyme Q biosynthetic defect and multiple respiratory chain deficiency, as detailed above in FIGS. 15 and 16, demonstrates their deficiency of NAD+ and corresponding increase in NADH:NAD+ redox balance relative to wild-type (B6) control mouse liver. (+) Epicatechin treatment in the animals' drinking water at 5 μg/mL to 10 μg/mL for 2 months did not correct this redox imbalance, highlighting the therapeutic need for combinatorial therapies of (+) epicatechin as a mitochondrial biogenesis agent together with NAD+ agonist therapies (such as niacin, niaciamide, or nicotinic acid) to replete their NAD+ deficiency that is common in complex I-related mitochondrial disorders. Biochemical studies are shown both by sex and all grouped together, where n=number of animals studied per group.

FIG. 18: Zebrafish swimming activity that is reduced in AB wild-type fish when exposed on 7 dpf to high-dose (70 nMol) rotenone, which is a potent mitochondrial complex I inhibitor, was synergistically rescued by combinatorial pre-treatment for 48 h with 100 nMol (−) epicatechin (EP03) plus 2 nMol 11-β-hydroxypregnenolone (EP06). Combinatorial therapies of several other low-dose nanomoloar range flavonoid treatments did not show similar synergistic effect in this model. EP01, (+) Epicatechin. EP07, 11-hydroxyprogesterone. **, p<0.01. Each symbol conveys activity of one well with n=8 animals/well in a 96-well plate, as quantified in dark-cycle analysis in the Zebrabox (Viewpoint).

### DETAILED DESCRIPTION

Primary mitochondrial respiratory chain (RC) disease is highly heterogeneous in etiologies and phenotypes, with causal pathogenic variants (mutations) now recognized in more than 350 different genes across both nuclear and mitochondrial genomes, following all Mendelian and maternal inheritance patterns. This new genomic understanding represents a transformative explosion in our understanding of mitochondrial RC disease etiologies and biochemical mechanisms. More than 5-fold increased identification of mitochondrial disease genes has occurred over the last decade, with likely hundreds more to be recognized—indeed, over half of causal gene disorders were identified in the past 6 years. Secondary mitochondrial RC dysfunction is also now widely recognized to occur in a host of common disorders, from neurodegenerative diseases such as Parkinson's and Alzheimer Disease, to complex phenotypes like metabolic syndrome, aging, sepsis, and ischemia-reperfusion injury after cardiac arrest or stroke. Sharing the basic underlying phenomenon of energy failure, RC disorders involve an impressively diverse spectrum of functional deficiencies that clinically present across central, peripheral, and autonomic nervous systems, skeletal muscle, heart, gastrointestinal tract, kidney, vision, hearing, hematologic, endocrine, and immune systems. Remarkably, each mitochondrial disease patient suffers on average 16 (range 7-35) major medical problems, which may involve any organ(s) and onset at any stage in their lifespan from birth through old age. With a collective minimal prevalence of 1 in 4,300, mitochondrial disease inflicts high health care burden and cost.

To characterize and optimize efficacious and non-toxic individual and multi-drug treatments for major respiratory chain (RC) disease subtypes, we have employed C. elegans (worm, invertebrate animal) and D. rerio (zebrafish, vertebrate) animal models of major RC disease. Whole animal survival and health assessments performed in worms included lifespan analysis application of an integrated, rapid screen of worm development (WormScan), as well as a range of neuromuscular activity analysis.

C. elegans gas-1(fc21) mutant RC worms are a robust and well-established model of mitochondrial disease that have ˜70% reduction of RC complex I function due to a homozygous mutation in the nuclear encoded NDUFS2 complex I subunit. These animals also have a ˜50% reduction in their lifespan, increased mitochondrial oxidant stress, as well as reduced mitochondrial membrane potential and mitochondrial content.

There is currently no cure or FDA-approved therapy for any mitochondrial disease, since little is known about downstream biochemical and physiologic abnormalities that contribute to their diverse clinical manifestations. Existing therapies are nonspecific, symptom management-based, and non-curative.

Mitochondrial complex I deficiency is due to limited structure, assembly, or function (deficiency) of a very large protein complex called complex I. Complex I is found in cell structures called mitochondria, which convert the energy from food into a form that cells can use. Complex I is the first of five mitochondrial respiratory chain complexes that carry out a multi-step process called oxidative phosphorylation, through which cells derive much of their energy.

### Zebrafish Assays

Using CRISPR-Cas9 technology, we have generated ndufs2-knockout fish lines (NDUFS2 p.R290K). We have prepared and characterized diverse mitochondrial diseases using CRISPR/Cas9 to generate knockout lines for inducing gross animal abnormalities and swimming behavior in a series of stress-response assays. This approach is valid as human mitochondrial diseases often have stress-responsive metabolic dysfunction and functional phenotypes that are not readily apparent at baseline. We have assessed gross development, survival, organ-level morphology, heart rate, swimming activity (dark activity induced), and integrated neurobehaviors of tap and touch response at baseline and in response to stressors. Animals are screened in the zebrabox high-throughput behavioral analysis system both at baseline and after exposure to stressors including nutrient stress (over and underfeeding), cold and hot temperature stress, infection mimetics such as LPS, and additional mitochondrial inhibitor stresses (rotenone, chloramphenicol, azide, or potassium cyanide). Once a stressor is identified that reliably impairs swimming behavior in each mitochondrial disease mutant larvae model, we used the stressor-model to test a multi-drug panel, and optimal combinations, as were previously identified in C. elegans as described above. Lead treatment effects in each zebrafish model can be validated by assessing mitochondrial physiology in diverse organs by confocal analysis and by fluorescence microscopy quantitation of Mitotracker Green/TMRE co-injected dyes co-injected into the early embryo yolk sac, or by analysis of stable genetic fluorescent lines that indicate mitochondrial amount. Biochemical effects will be assessed by spectrophotometric assay of ETC activities, HPLC-ECD analysis of glutathione (GSH and GSSG) oxidative stress, and GC/MS based metabolomics analyses, as appropriate.

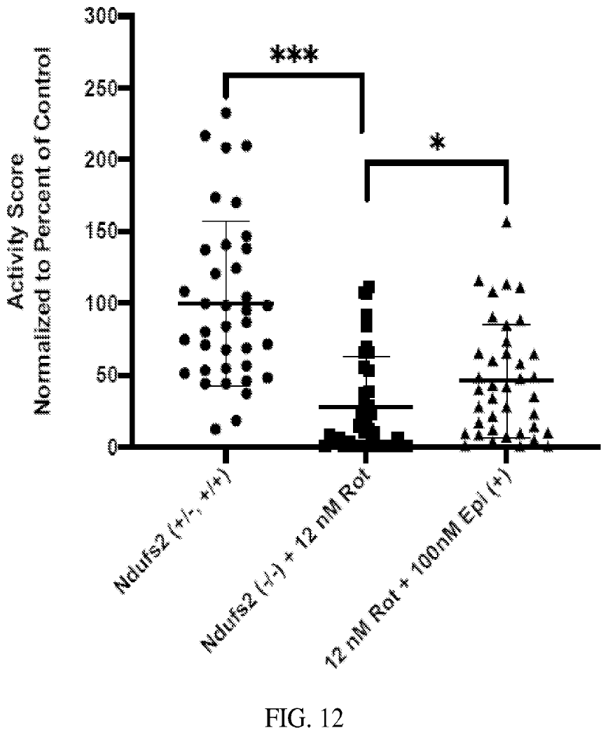

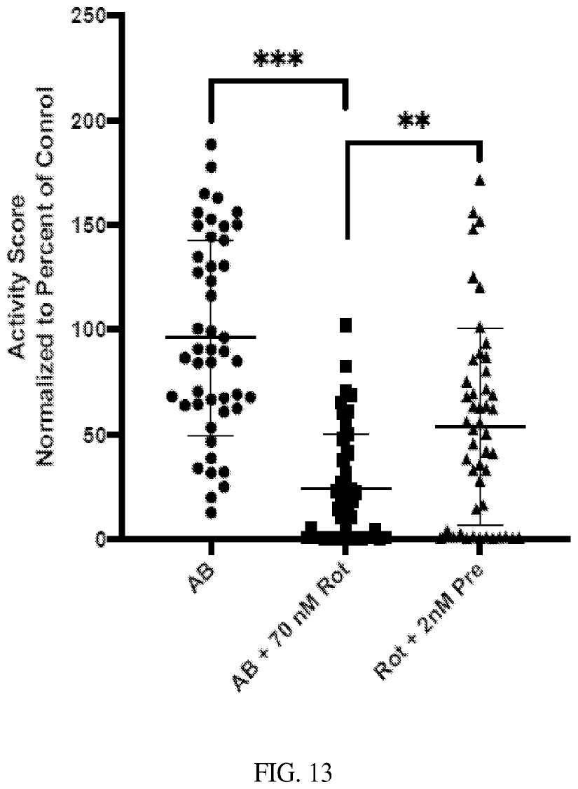

Using the Rotenone model which induces brain death in zebrafish, we tested various drugs alone and in combination and identified protective combination that should have efficacy for the treatment of mitochondrial disorders. While Rotenone is exemplified in FIGS. 12 and 13), other specific RC stressors that exacerbate phenotypes in cell and animal models of mitochondrial RC disease could be employed. These include for example, sodium azide, chloramphenicol, and potassium cyanide. Phenotypes assessed can include without limitation, swimming activity or swimming fatigue, organ structural impairment or dysfunctional mitochondria leading to gray-colored brain on microscopy analysis (indicating brain death),), heart rate, startle (tap) response (stimulus on plate), and touch response (stimulus on animal). Additional ex vivo analyses can be performed to assess effects of the mutations or therapies on diverse aspects of mitochondrial physiology (oxidative phosphorylation capacity, electron transport chain enzyme activity levels, mitochondrial amount, metabolite levels, ATP levels, etc).

These animal models and human cell lines have been used to advantage for characterizing new efficacious agents useful for the treatment of mitochondrial and other respiratory chain disorders.

### Definitions

11β-Hydroxyprogesterone (11β-OHP), (also known as 21-deoxycorticosterone, of 11β-hydroxypregn-4-ene-3,20-dione), is a naturally occurring, endogenous steroid and derivative of progesterone. It is a potent mineralocorticoid.

11α-Hydroxyprogesterone (11α-OHP), or 11α-hydroxypregn-4-ene-3,20-dione is an endogenous steroid and metabolite of progesterone. It is a weak anti-androgen, and is devoid of androgenic, estrogenic, and progestogenic activity.

(−)-Epicatechin, the most abundant flavanol present in cacao, appears to largely mediate the health effects ascribed to the consumption of this product. The two isoforms of epicatechin, (−) and (+), structurally resemble or mimic 11-β-hydroxypregnenolone, a naturally occurring sterol recently shown to be a potent inducer of mitochondrial biogenesis (mtB).

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, C. elegans, zebrafish, mice, rats, hamsters, and primates.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

A “genetic or protein alteration” as used herein, includes without limitation, naturally occurring mutations, chemically induced mutations, genetic alterations generated via introduction of siRNA, antisense oligonucleotides and CRISPR-CAS9 targeted gene constructs. Protein alterations can be generated via pharmacological inhibition or modification of proteins involved in mitochondrial respiratory chain function.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented.

As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of a mitochondrial disease associated gene.

As used herein, “mitochondrial related disorders” related to disorders which are due to abnormal mitochondria structure or function, such as for example, a mitochondrial genetic mutation, enzyme pathways, etc. Examples of disorders include and are not limited to: loss of motor control, muscle weakness and pain, gastrointestinal disorders and swallowing difficulties, poor growth, cardiac disease, liver disease, diabetes, respiratory complications, seizures, visual/hearing problems, lactic acidosis, developmental delays and susceptibility to infection. The mitochondrial abnormalities give rise to “mitochondrial diseases” which include, but not limited to: AD: Alzheimer's Disease; ADPD: Alzheimer's Disease and Parkinson's Disease; AMDF: Ataxia, Myoclonus and Deafness, CIPO: Chronic Intestinal Pseudo-obstruction with myopathy and Opthalmoplegia; CPEO: Chronic Progressive External Ophthalmoplegia; DEAF: Maternally inherited Deafness or aminoglycoside-induced Deafness; DEMCHO: Dementia and Chorea; DMDF: Diabetes Mellitus & Deafness; Exercise Intolerance; ESOC: Epilepsy, Strokes, Optic atrophy, & Cognitive decline; FBSN: Familial Bilateral Striatal Necrosis; FICP: Fatal Infantile Cardiomyopathy Plus, a MELAS-associated cardiomyopathy; GER: Gastrointestinal Reflux; KSS Kearns Sayre Syndrome LDYT: Leber's hereditary optic neuropathy and Dystonia; LHON: Leber Hereditary Optic Neuropathy; LIMM: Lethal Infantile Mitochondrial Myopathy; MDM: Myopathy and Diabetes Mellitus; MELAS: Mitochondrial Encephalomyopathy, Lactic Acidosis, and Stroke-like episodes; MEPR: Myoclonic Epilepsy and Psychomotor Regression; MERME: MERRF/MELAS overlap disease; MERRF: Myoclonic Epilepsy and Ragged Red Muscle Fibers; MHCM: Maternally Inherited Hypertrophic CardioMyopathy; MICM: Maternally Inherited Cardiomyopathy; MILS: Maternally Inherited Leigh Syndrome; Mitochondrial Encephalocardiomyopathy; Mitochondrial Encephalomyopathy; MM: Mitochondrial Myopathy; MMC: Maternal Myopathy and Cardiomyopathy; Multisystem Mitochondrial Disorder (myopathy, encephalopathy, blindness, hearing loss, peripheral neuropathy); NARP: Neurogenic muscle weakness, Ataxia, and Retinitis Pigmentosa; alternate phenotype at this locus is reported as Leigh Disease; NIDDM: Non-Insulin Dependent Diabetes Mellitus; PEM: Progressive Encephalopathy; PME: Progressive Myoclonus Epilepsy; RTT: Rett Syndrome; SIDS: Sudden Infant Death Syndrome.

### Pharmaceutical Formulations

Where clinical applications are contemplated, pharmaceutical compositions will be prepared in a form appropriate for the intended application. Generally, this will entail preparing compositions (e.g., expression vector) that are essentially free of pyrogens, as well as other impurities that could be harmful to humans or animals.

One will generally desire to employ appropriate salts and buffers to render drugs stable and allow for uptake by target cells. Aqueous compositions of the present disclosure comprise an effective amount of the drug dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

The active compositions of the present disclosure may include classic pharmaceutical preparations. Administration of these compositions according to the present disclosure may be via any common route so long as the target tissue is available via that route. This includes oral, nasal, or buccal, as well as through nasal feeding tubes or gastrostomy or jejunual ports and tubes that are commonly needed in primary mitochondrial disease patients. Alternatively, administration may be by intradermal, subcutaneous, intramuscular, intraperitoneal or intravenous injection. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present disclosure generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic saline (NaCl) solution and either added to 1000 ml of hypodermoclysis fluid or injected at the proposed site of infusion. (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics Standards.

### Therapies

In another embodiment, combinatorial treatment of mitochondrial disease is contemplated. Combinations may be achieved by treating patients with a single composition or pharmacological formulation that includes two or more agents, or by treating the patient with distinct compositions or formulations, at the same time, wherein each composition includes a distinct agent. Alternatively, the various agents may be given in a staggered fashion ranging from minutes, to hours, to weeks. In such embodiments, one would generally ensure that the period of time between each delivery was such that the agents would still be able to exert an advantageously combined effect on the cell or subject. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

By way of illustration, where 11β-Hydroxyprogesterone is “A”, (+) epicatechin is “B.” and a third agent is “N-acetyl-cysteine,” the following permutations are exemplary: A/B/C B/A/C A/C/B B/C/A C/A/B C/B/A Other combinations wherein multiple administrations of one or more agents are likewise contemplated.

Furthermore, multiple administrations of the cocktail itself are contemplated, such as in an ongoing or chronic basis. The administrations may be twice daily, daily, twice weekly, weekly, every other week, or monthly. They may also be administered for therapeutic purposes to mitochondrial disease patients who are acutely decompensating on a continual or more frequent basis in an acute medical setting (emergency department, intensive care unit, etc).

In another aspect, the present disclosure provides compositions comprising one or more of compounds as described above and an appropriate carrier, excipient or diluent. The exact nature of the carrier, excipient or diluent will depend upon the desired use for the composition, and may range from being suitable or acceptable for veterinary uses to being suitable or acceptable for human use. The composition may optionally include one or more additional compounds.

When used to treat or prevent such diseases, the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as steroids, MAPK-modulators, membrane stabilizers, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making, levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically. The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, oral, topical, ocular, buccal, systemic, nasal, injection, transdermal, rectal, vaginal, etc., or a form suitable for administration by inhalation or insufflation.

For oral administration, the pharmaceutical compositions may take the form of, for example, lozenges, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For buccal administration, the compositions may take the form of tablets or lozenges formulated in conventional manner.

For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC.sub.50 of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active metabolite compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective local dosages without undue experimentation.

### Materials and Methods

Mitochondrial oxidant burden (MitoSOX Red), membrane potential (tetramethylrhodamine ethyl ester, TMRE), and mitochondrial content (MitoTracker Green FM, MTG) were performed in C. elegans at 20° C. using in vivo terminal pharyngeal bulb relative fluorescence microscopic quantitation. Briefly, synchronous populations of Day 0 young adults were moved to 35 mm NGM plates spread with OP50 E. coli, a desired drug treatment (e.g., different concentrations of (+) epicatechin in combination with other agents (galactose) or buffer control (S-basal/water for all other drugs) was performed on NGM plates. Simultaneously with the drug treatments, worms were treated with either 10 mM MitoSOX Red (matrix oxidant burden), 100 nM TMRE (mitochondrial membrane potential), or 2 μM MitoTracker Green FM (mitochondria content) for 24 h. The next day, worms were transferred with a pick onto 35 mm agar plates spread with OP50 E. coli without dye for 1 h to allow clearing of residual dye from the gut. Worms were then paralyzed in situ with 5 mg/ml levamisole. Photographs were taken in a darkened room at 160.times. magnification with a Cool Snap cf2 camera (Nikon, Melville, N.Y.). A CY3 fluorescence cube set (MZFLIII, Leica, Bannockburn, Ill.) was used for MitoSOX and TMRE. A GFP2 filter set (Leica) was used for MitoTracker Green FM. Respective exposure times were 2 s, 320 ms, and 300 ms for each of MitoSOX, TMRE, and MitoTracker Green FM. The resulting images were background subtracted, and the nematode terminal pharyngeal bulb was manually circled to obtain mean intensity of the region by using Fiji Is Just ImageJ. Fluorescence data for each strain were normalized to its same day control to account for day-to-day variation. A minimum of 3 independent experiments of approximately 50 animals per replicate were studied per strain per dye. The significance of the difference in the mean fluorescence intensity between strains under different experimental conditions was assessed by mixed-effect ANOVA, which analyzes potential batch effect due to samples being experimentally prepared, processed, and analyzed on different days by including a batch random effect in the model. A statistical significance threshold was set at P<0.05. All statistical analyses were performed in SAS 9.3.

Neuromuscular activity, and lifespan were quantified in the C. elegans gas-1(fc21) ndufs2^{-/-} mutants. A semi-automated screening method (Mathew et al, 2016) was used to test drug treatment effects on an integrated C. elegans health endpoint of fecundity, brood size, and behavior.

Human fibroblasts were studied from a subject harboring a 1067del (p.Gly356Alafs*15) nonsense mutation in the maternal FBXL4 allele and a c. 1790A>C (p.GLn597Pro) missense mutation in the paternal FBXL4 allele (Gal et al, 2013). Fibroblasts were cultured in DMEM (1 g/L glucose, 0.8 g/L L-Glutamine, 110 mg/L Sodium Pyruvate). Light, fluorescence, confocal microscopy and transmission electron microscopy (TEM, Lavorato et al. 2017) methods were used to analyze proband fibroblasts and mitochondrial morphology at baseline and following metabolic stress induced by incubating cells for 48 hours in glucose/uridine-free media. Mitotracker green was used for fluorescence microscopy, Tom20 Antibody (Santa Cruz) and DAPI was used for confocal microscopy.

The following examples are provided to illustrate certain embodiments of the invention. They are not intended to limit the invention in any way.

### Example I

Primary mitochondrial respiratory chain (RC) disease afflicts at least 1 in 4,300 people with multi-system manifestations for which there currently are no proven effective treatment other than empiric antioxidants and cofactors. Targeting precise regulatory molecules in the integrated nutrient-sensing signaling network (NSSN) that sense and coordinate the global cellular dysfunction occurring in RC disease (1, 2) may offer a personalized path to alleviate or prevent patient morbidity in RC disease.

(−) Epicatechin is commercially available for human consumption, while (+) epicatechin is more difficult to make from the naturally occurring racemic mixture. (+) Epicatechin is a naturally-occurring flavonoid in chocolate, tea, and guarana that presents a particularly appealing therapeutic agent to potentially “reset” pathologic NSSN alterations that are caused by RC dysfunction and ultimately improve cellular and mitochondrial health in RC disease. Preliminary studies of its less potent enantiomer. (−) Epicatechin in diabetic myopathy and cisplatin nephropathy (3) have suggested epicatechin effects may improve mitochondrial mass, oxidative balance, and structure.

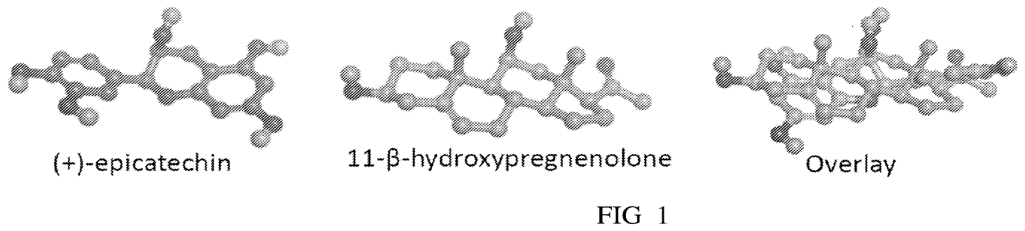

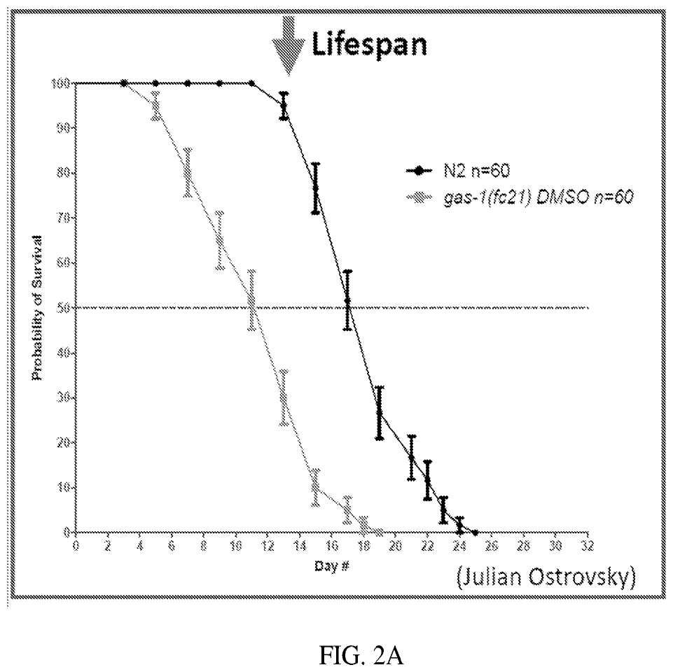

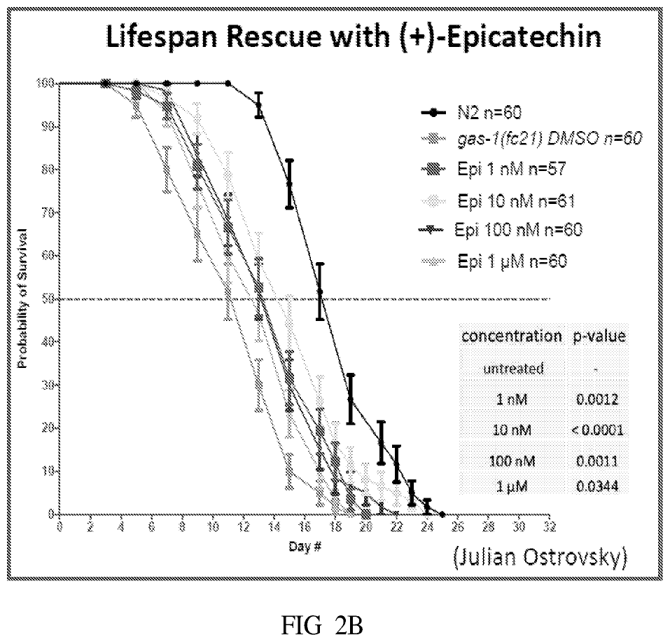

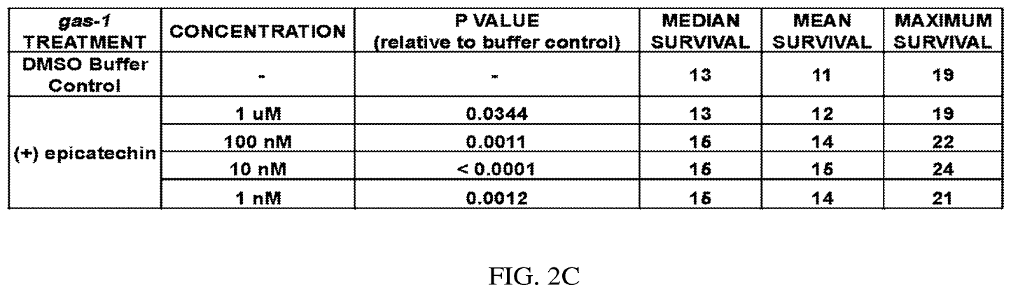

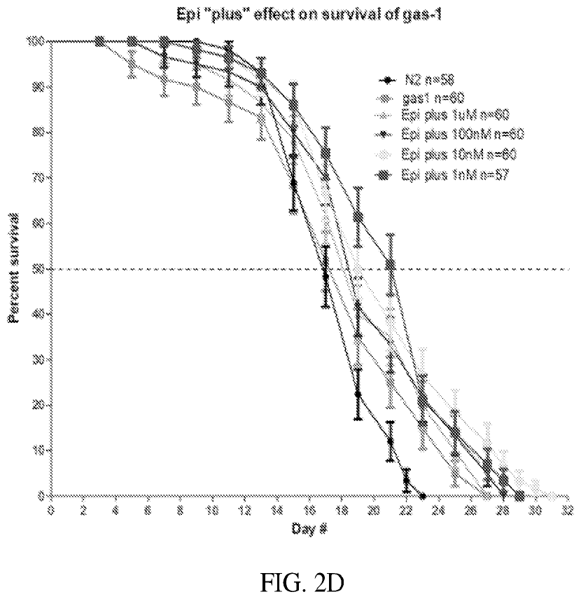

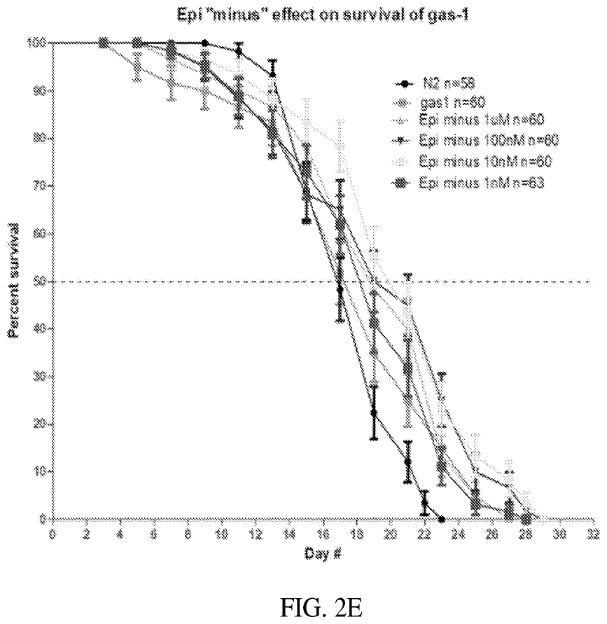

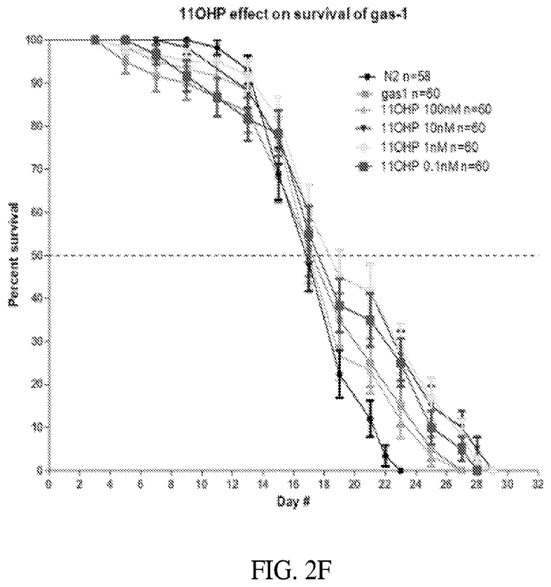

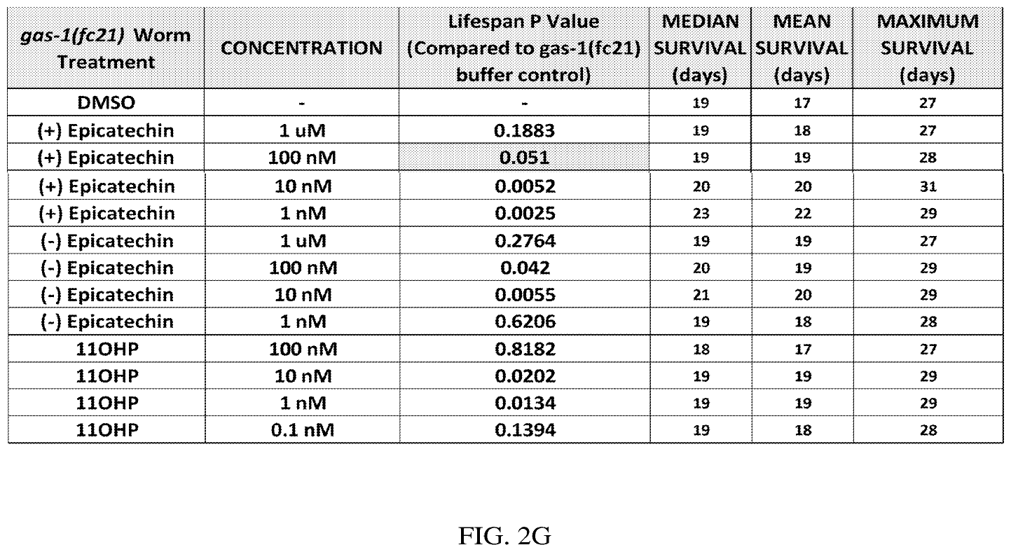

FIG. 1 shows an overlay of the two isoforms of this molecule. (−) Epicatechin and (+) Epicatechins act through mimicry of the endogenous mitochondrial steroid 11-β-hydroxypregnenolone (11-OHP), a potent inducer of mitochondrial biogenesis. C. elegans nematode strains studied included wild-type (N2 Bristol) and gas-1(fc21) mutants. The data in FIGS. 2A and 2B reveal that the short lifespan of gas-1(fc21) mutants was significantly rescued with 10 nM (+) Epicatechin. Relative to N2 Bristol wild-type worms (black line), median and maximal lifespans of short-lived gas-1 mutant worms (red line) are significantly improved with (+) epicatechin, where the greatest effect was seen with 10 nM treatment (green line). FIG. 2C shows a summary of the data in FIGS. 2A and 2B. FIGS. 2D, 2E and 2F are graphs show a side-by-side comparison of (+)-epicatechin, (−)-epicatechin and 11-0HP on gas-1(fc-21) worm survival. The data is tabulated in FIG. 2G.

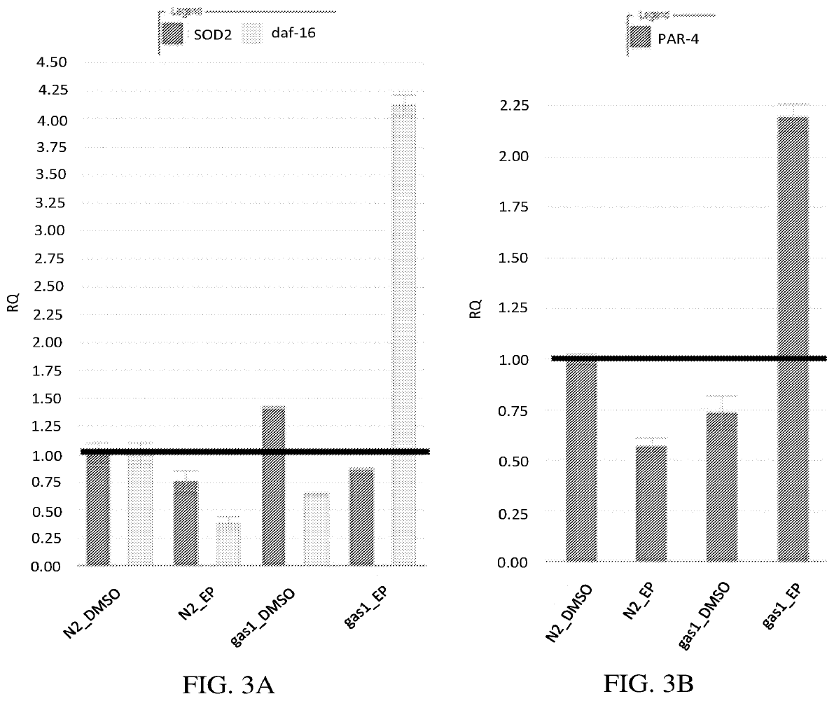

FIGS. 3A-3B show that (+) Epicatechin effects C. elegans signaling via NSSN gene expression. Recently the intracellular signaling system for (+) epicatechin has been found to include activation of NSSN central nodes, including activation of AMPK and SIRT. It is already evident that epicatechin therapy in some diseases can favorably alter the activities of central NSSN nodes, such as AMPK, that are known to regulate cell proliferation, autophagy, and mitochondrial proliferation. These preliminary findings are suggestive that using epicatechin to “reset” alterations in the NSSN that sense and coordinate the global cellular dysfunction occurring in RC disease may offer a path to alleviate or prevent morbidity in RC disease. FIG. 3C shows similar effects of (+)-epicatechin and 11-hydroxypregnenolone on RNAseq aggregated as KEGG pathways, with unique induction of MAPK pathway signaling. The pathways marked with a star can similarly be restored in gas-1(fc21) complex I deficient worms by combination therapy of glucose+nicotinic acid+N-acetylcysteine.

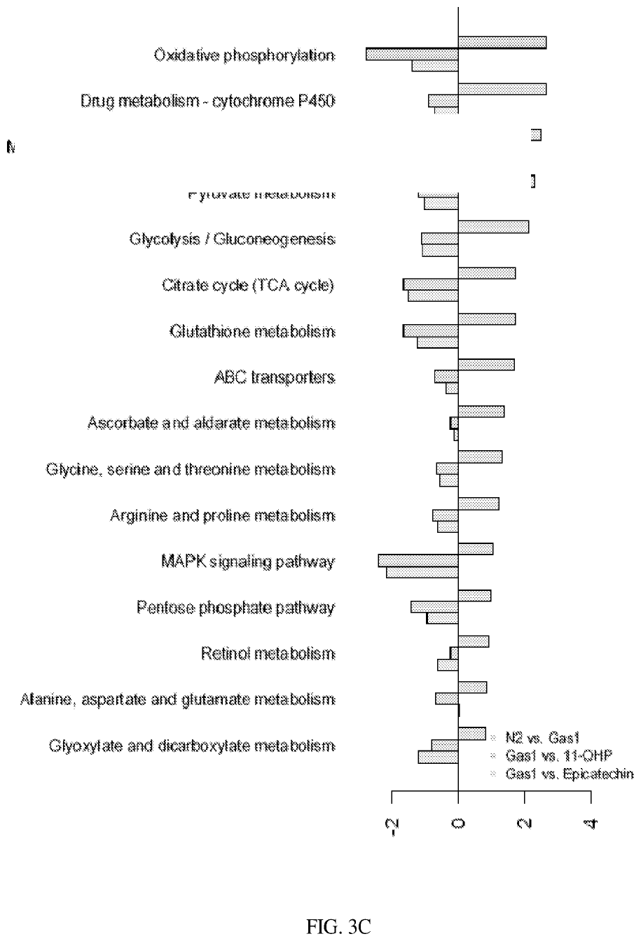

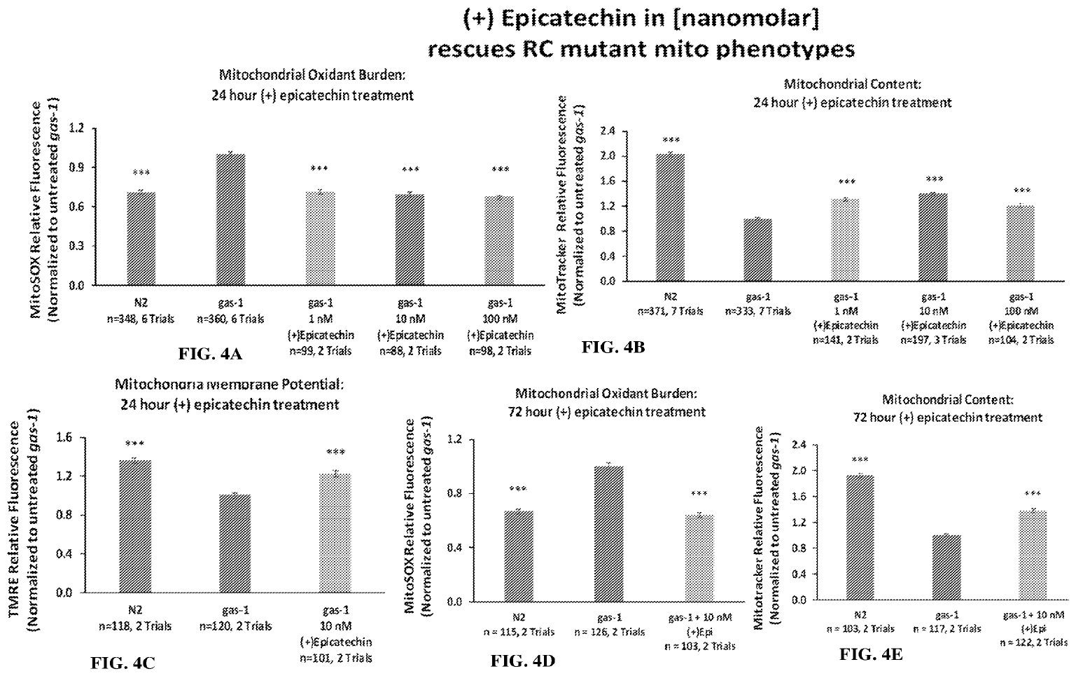

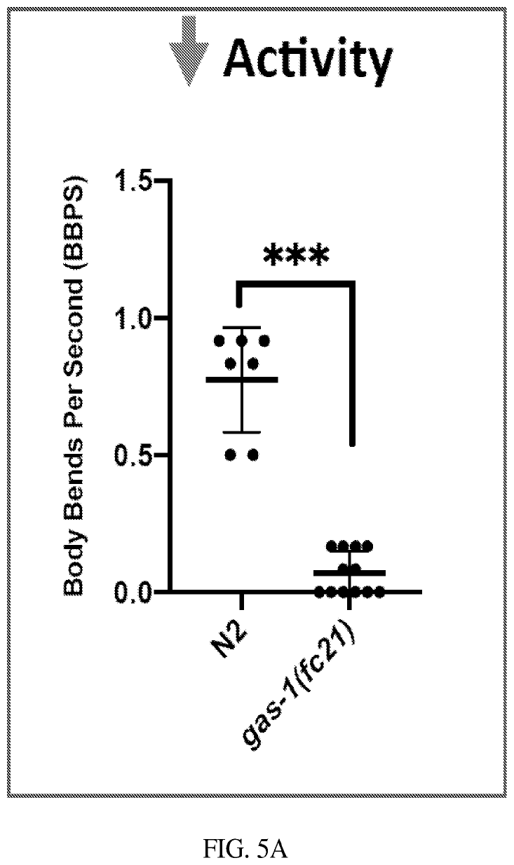

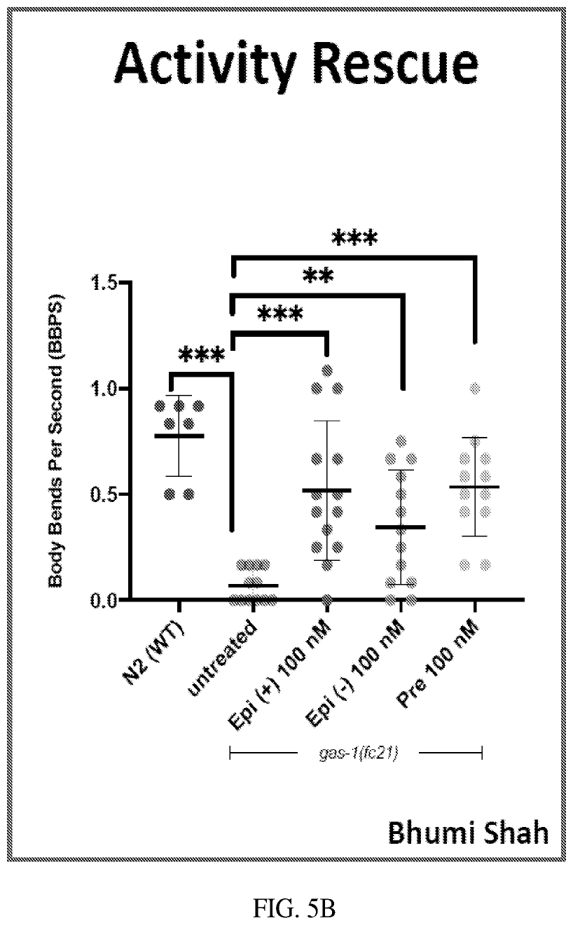

FIGS. 4A-4E show in vivo quantification of relative mitochondrial oxidant burden, mitochondrial content, and mitochondrial membrane potential in gas-1(fc21) complex I disease worms. Terminal pharyngeal bulb fluorescence analysis of MitoSox (FIG. 4A) and MitoTracker Green (FIG. 4B) were performed in 3-log order dose range of gas-1(fc21) worms treated for 24 hours with (+) epicatechin relative to untreated gas-1(fc21) and wild type (N2) worms, which indicated complete normalization of mitochondrial oxidant burden at all concentrations and significant improvements in mitochondrial content (most improved by 39% with 10 nM concentration) in RC deficient worms. Mitochondrial membrane potential was also significantly improved by 62% towards wild type levels with 10 nM (+) epicatechin for 24 hours in gas-1(fc21). FIG. 4C) Sustained effects were seen at 72 hours of treatment (with redosing every 24 hours) in terms of complete reversal of mitochondrial oxidant burden. FIG. 4D) and significant rescue of mitochondrial content (FIG. 4E) *** p<0.001 relative to untreated gas-1(fc21) worms. (+) Epicatechin also rescues body bend activity in gas-1(fc21) mutants. See FIG. 5. Notably, pretreatment with 100 nM 11-β-hydroxypregnenolone was also protective, as shown in FIG. 5B.

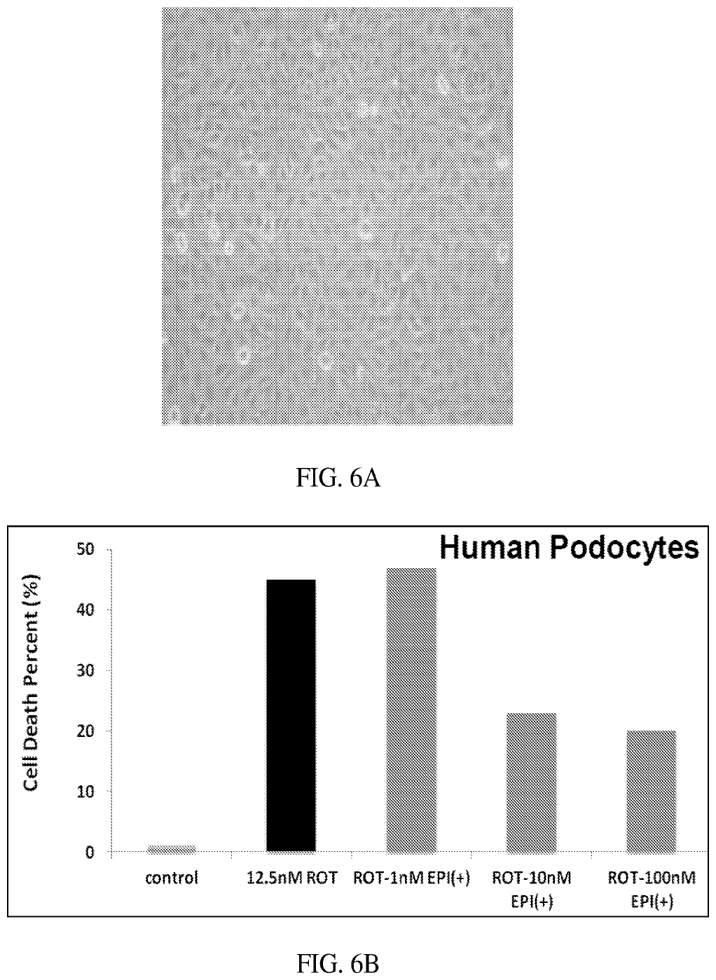

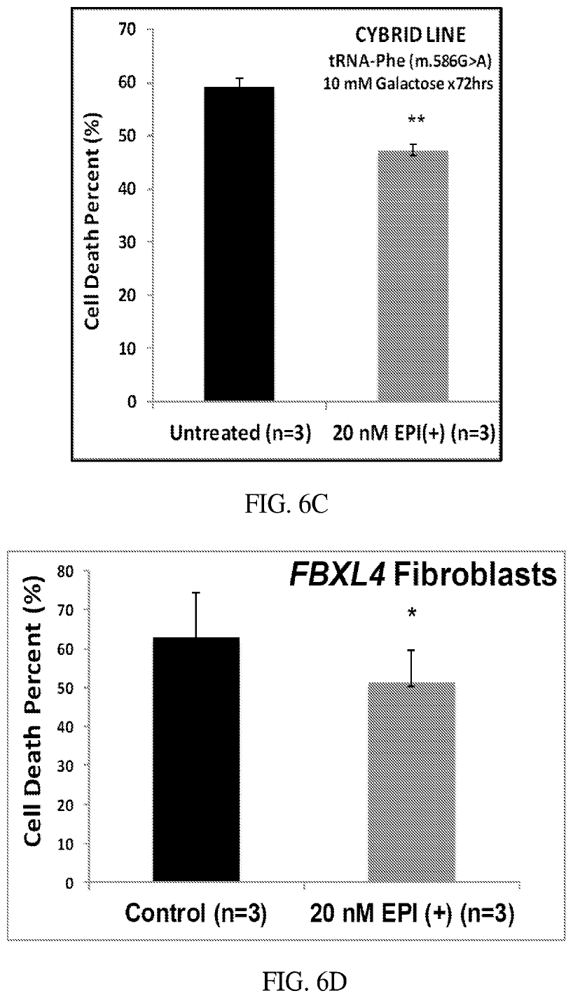

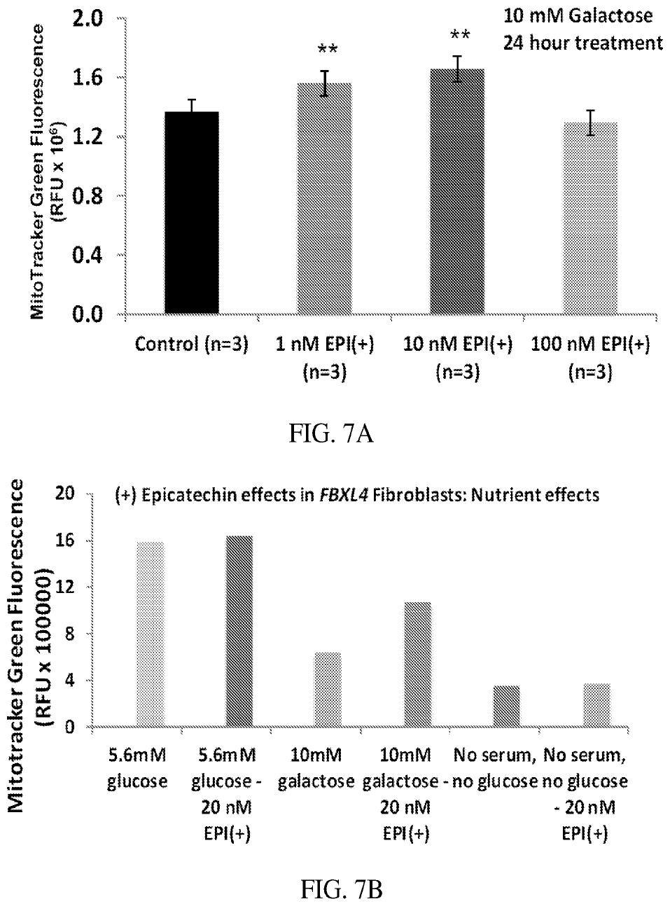

We also observed that (+) epicatechin modulates human cell viability and mitigates cell death in pharmacologic or genetic RC disease. FIGS. 6A-6C show human cell in vitro models of RC disease: Cell models studied included [1] Pharmacologic RC complex I inhibition with rotenone in healthy human podocytes; [2] trans-mitochondrial cybrid cell lines from a patient with tRNA-Phe mtDNA (m.586G>A) mutation (4); and [3] Fibroblast cell line from a patient with FBXL4-based mitochondrial disease (5). Cells were treated overnight with (+) Epicatechin in concentrations ranging from 1 nMol to 100 nMol. The data show that (+) Epicatechin improves mitochondrial mass in human FBXL4 patient fibroblasts. Moreover, nutrients such as galactose can potentiate beneficial (+) Epicatechin effects. See FIG. 7.

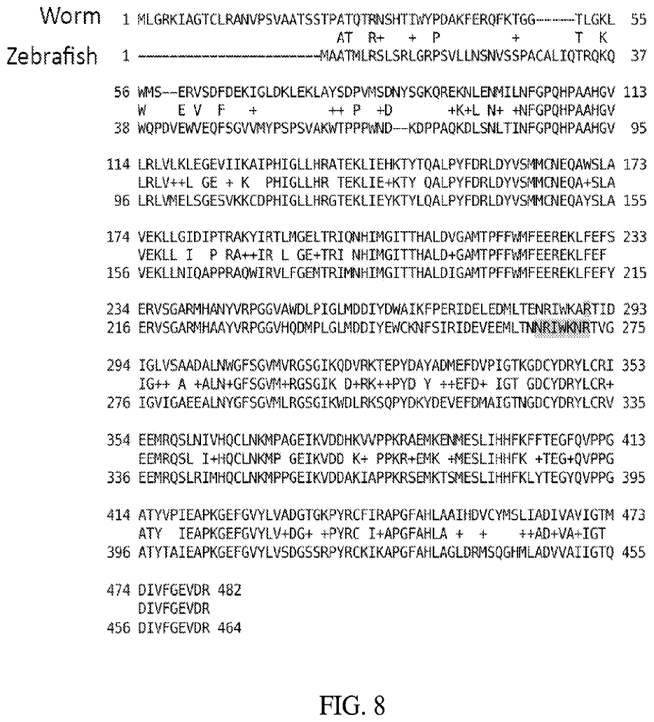

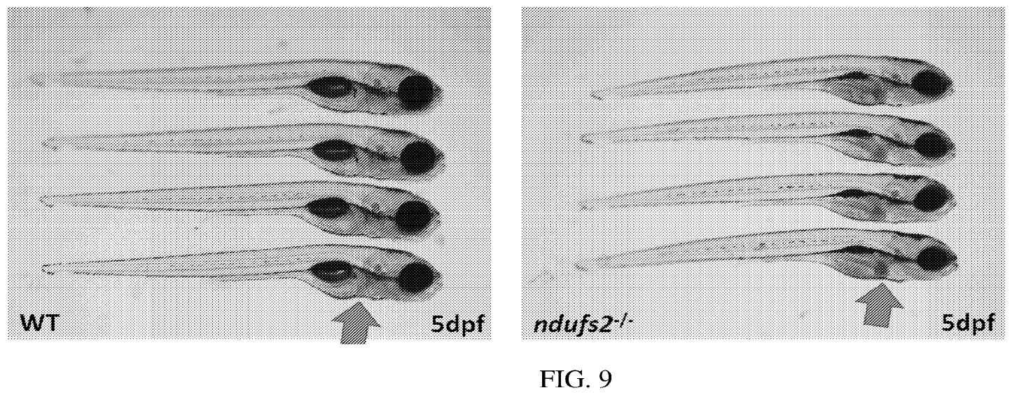

Finally, we also investigated the therapeutic potential of (+)-epicatechin, (−)-epicatechin, and 11-β-hydroxypregnenolone on Danio rerio (zebrafish, vertebrate) animal models of mitochondrial complex I disease. CRISPR/Cas9-generated ndufs2^{-/-} zebrafish were studied. Sec FIG. 8. C. elegans gas-1(fc21) worms have homozygous Arg→Lys mutation (p.R290K). Zebrafish ndufs2^{-/-} strain generated with CRISPR/Cas9 technology has a 16 base pair deletion which causes a frameshift mutation and premature stop codon. Both mutants are animal models for autosomal recessive NDUFS2-based complex I disease. The resulting ndufs2^{-/-} zebrafish phenotypes are shown in FIG. 9. Abnormal larval development evidenced by reduced yolk absorption is shown. These zebrafish mutants have no swim bladder and a grey round liver which is indicative of cell death. These zebrafish mutants exhibit abnormal neuromuscular behavior. For example, tap and touch responses are reduced moderately at 5 days post fertilization (dpf) and greatly at 7 dpf. Early death occurs at 9 dpf.

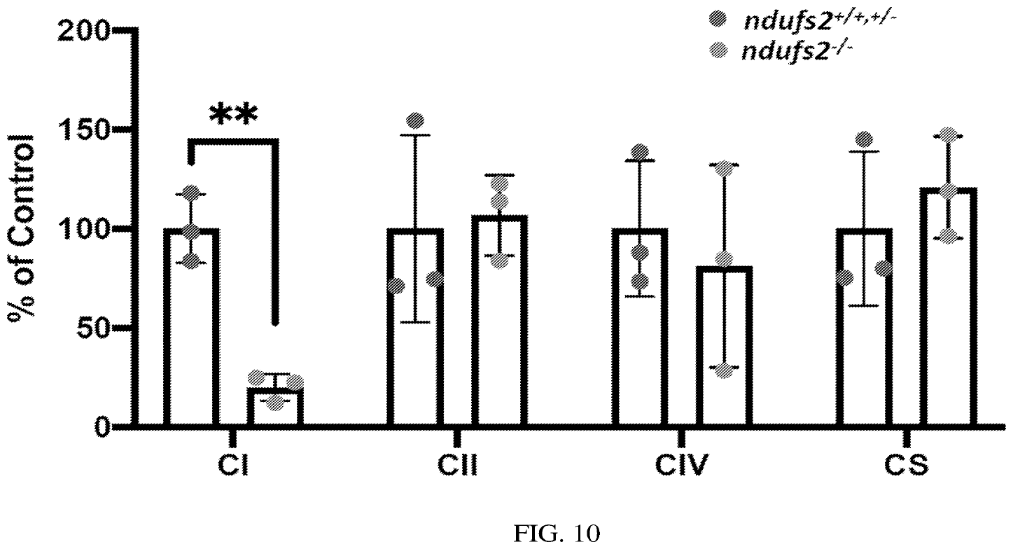

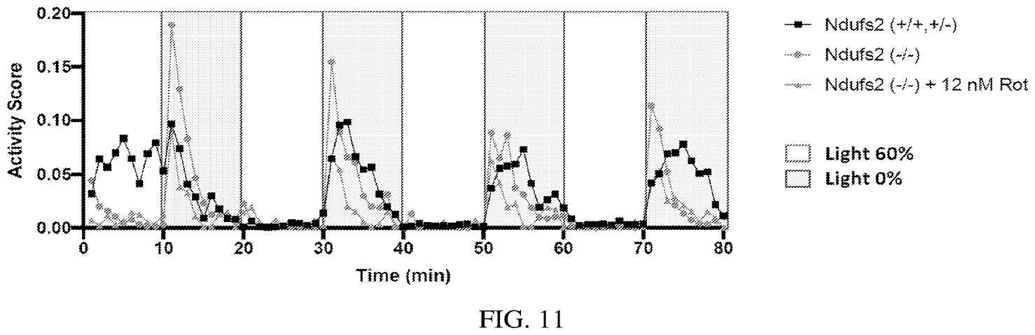

ndufs2^{-/-} zebrafish have selectively reduced respiratory chain complex I enzyme activity. CI activity was significantly reduced by 80% in larvae at 7 dpf (** P<0.01.). See FIG. 10. FIG. 11 shows that ndufs2^{-/-} zebrafish larvae display reduced dark-induced swimming activity upon exposure to low-dose complex I inhibition with rotenone for 4 hours at 7 dpf. These complex I mutants exhibit stressor hypersensitivity to low-dose acute complex I inhibition with the pharmacologic inhibitor rotenone.

Swimming activity (Zebrabox) was quantified in first five-minute periods of three consecutive dark cycles across 3 biological replicate experiments. Results are shown in FIG. 12. Activity score was normalized to percent of wild-type concurrent controls. 100 nM of (+)-epicatechin pre-treatment from 5 dpf significantly reduced dark-induced swimming impairment upon acute low-dose rotenone exposure at 7 dpf by ˜25% toward that of wild-type controls. AB (wild-type) zebrafish had reduced swimming activity with high dose (70 nM) rotenone exposure for 4 hours on 7 dpf. 2 nM of 11-β-hydroxypregnenolone pre-treatment from 5 dpf significantly reduced dark-induced swimming impairment upon acute high-dose rotenone exposure of AB larvae at 7 dpf by ˜25% toward that of AB controls. See FIG. 13.

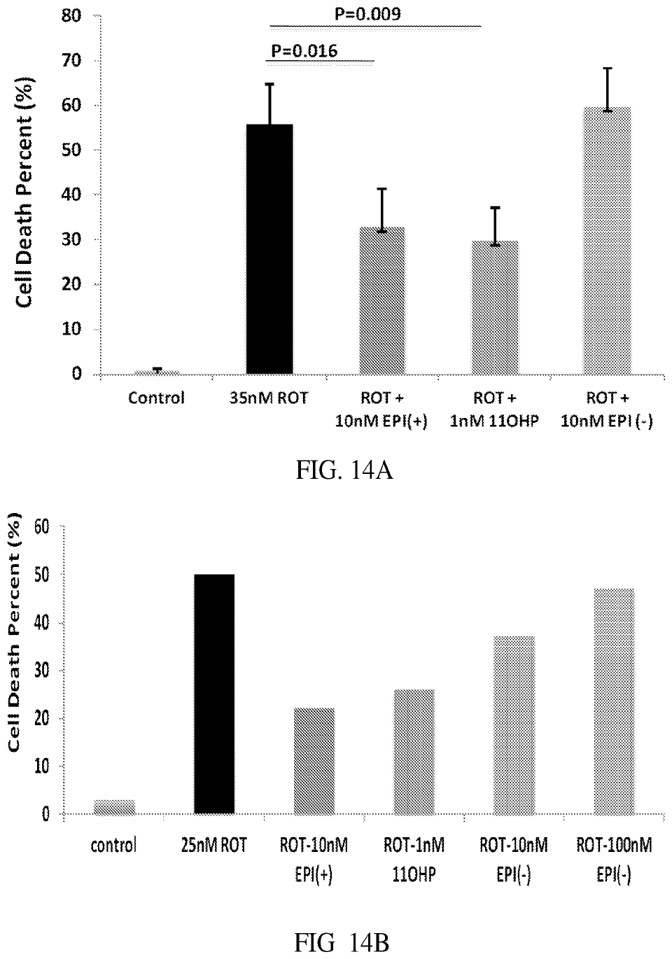

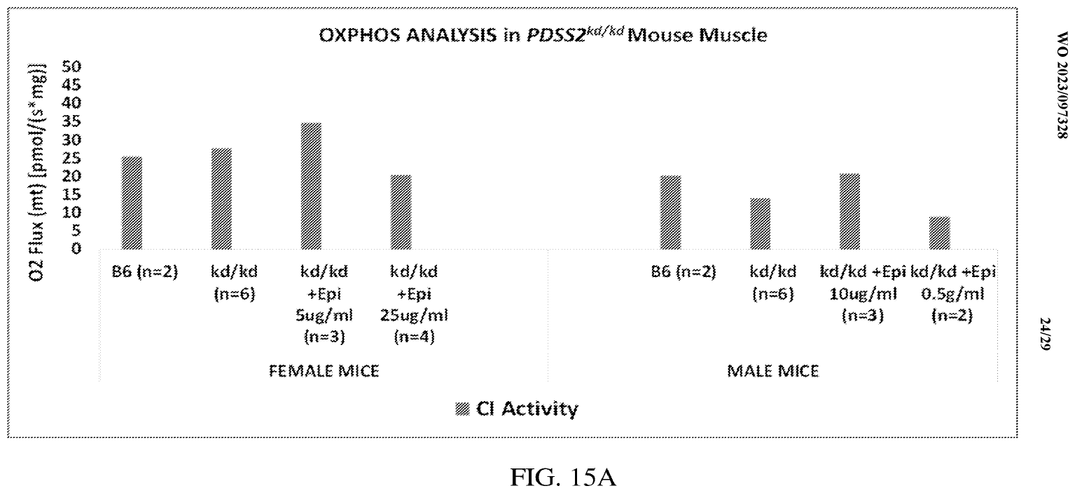

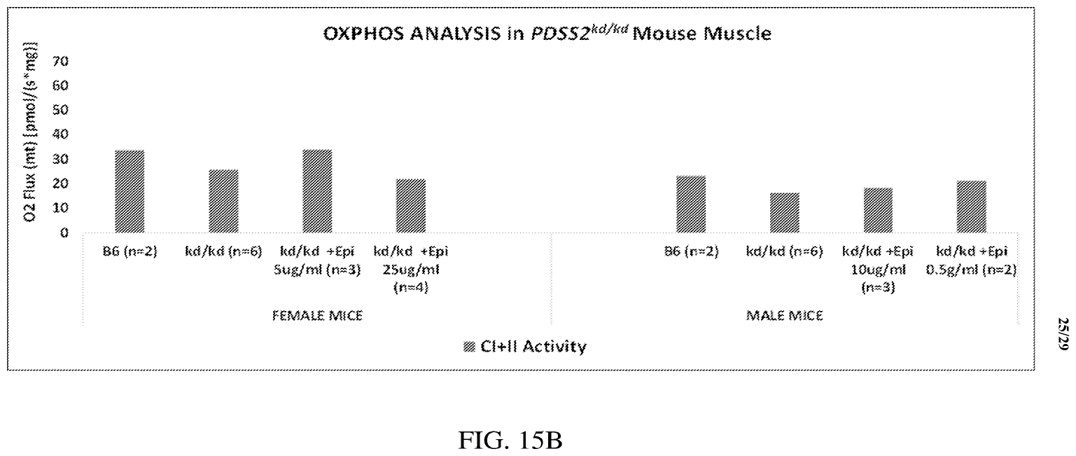

We also analyzed the effects of (+) epicatechin and (−) epicatchin in human cells treated with a potent RC complex I inhibitor (rotenone) and note that (+) epicatechin treated cells show clear improvement by approximately 50 percent in cell viability at very low (10 to 100 nanomolar) (+) epicatechin concentration (FIG. 14A-B). In addition, fibroblasts from a mitochondrial disease patient with RC complex I and III deficiency and mtDNA depletion caused by an FBXL4 gene mutation, showed a significant increase in cellular mitochondrial content (p<0.01) as measured by FACS analysis after mitotracker green overnight incubation, with an increase by 14% and 21% with 1 and 10 nM (+) epicatechin, respectively, compared compared to baseline (data not shown).

Further analysis of integrated mitochondrial respiratory chain capacity was performed by polarography in freshly isolated skeletal muscle using a permeabilized tissue protocol with malate as the complex I substrate, and malate+succinate as the complex I+II substrates, together with high ADP per standard protocol (Oxygraph 2k, Oroboros Instruments). N=2-6 animals/condition, as detailed. B6 control animals were sacrificed between 127 and 137 days of life. Pdss2^{kd/kd} mice were fed (+) Epicatechin ad libitum from approximately 90-120 days of life (when symptoms of renal glomerular disease were already present based on frank albuminuria) until sacrifice at approximately 150-190 days of life. As anticipated, reduced complex I+II respiratory capacity was seen in Pdss2^{kd/kd} missense mutant mice that have impaired coenzyme Q biosynthesis (which is needed for complex I+II activities) relative to wild-type (B6) control animals in both conditions for male mice and in the complex I+II activity for female mice. A consistent trend was seen of increased mitochondrial complex I and complex I oxphos capacity as well as increased mitochondrial complex I+II oxphos capacity with 5 μg/mL to 10 μg/mL (+) epicatechin oral treatment in their drinking water for 2 months after the onset of renal disease.

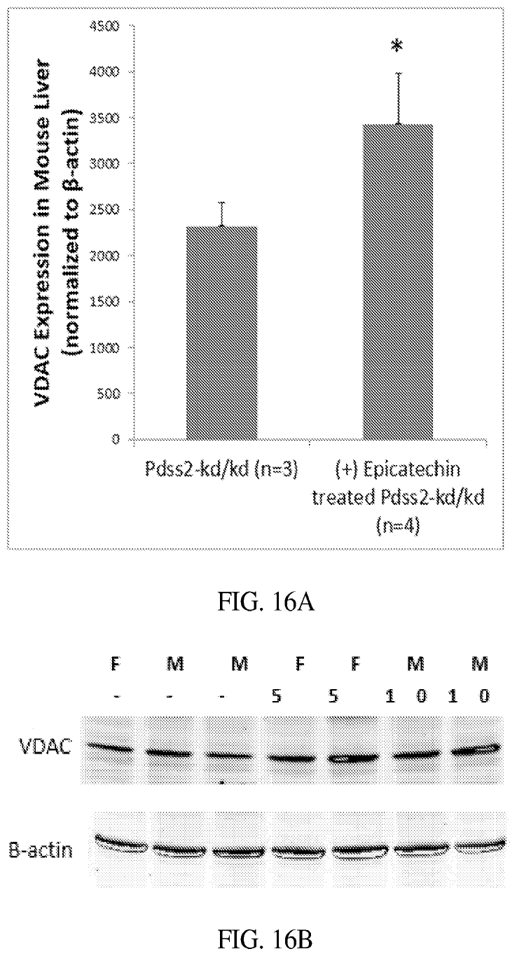

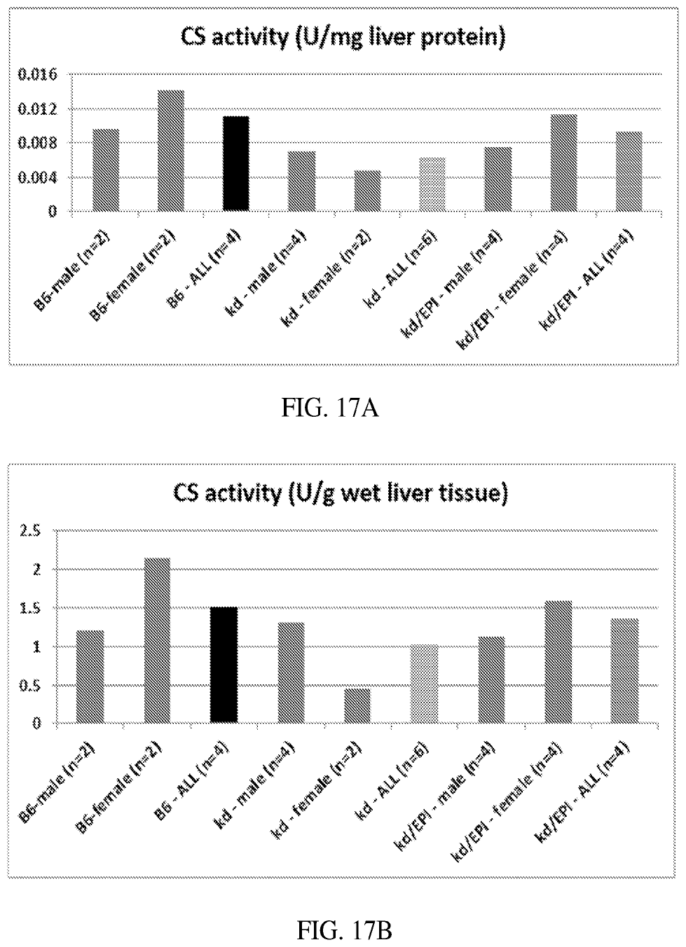

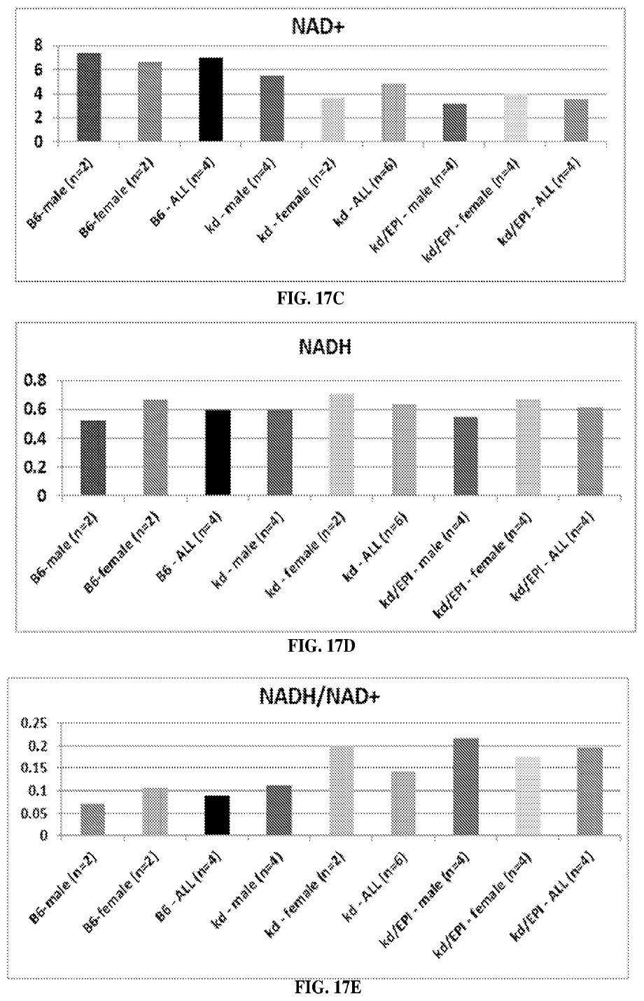

FIGS. 16A and 16B show that a significant increase in VDAC (porin) protein expression was measured as an outer mitochondrial membrane marker of mitochondrial amount in liver of (+) epicatechin fed Pdss2^{Kd/Kd} mice relative to untreated mutant controls (*, p<0.05). Untreated mice included 2M and 1F. (+) Epicatechin treated mice included two females treated with 10 μg/mL and 2 males treated with 5 μg/mL for approximately 2 months beginning at day of life 98-115. FIG. 16A is a graph quantifying the western blotting results shown in FIG. 16. In addition to VDAC increase, FIGS. 17A and 17B depict analyses of liver citrate synthase (CS) activity to provide further evidence that directly support the occurrence of mitochondrial depletion in Pdss2^{Kd/Kd} mice relative to wild-type (B6) animals (irregardless of sex), and the consistent biologic effect of (+) epicatechin at 5 to 10 μg/μL in drinking water to increase mitochondrial content in Pdss2^{Kd/Kd} mice. However, as shown in FIG. 17C-17D. (+) epicatechin treatment in the animals' drinking water at 5 μg/mL to 10 μg/mL for 2 months did not correct their NAD+ deficiency NADH/NAD+ redox imbalance, highlighting the therapeutic need for combinatorial therapies of (+) epicatechin as a mitochondrial biogenesis agent together with NAD+ agonist therapies (such as niacin, niaciamide, or nicotinic acid) to replete their NAD+ deficiency that is common in complex I-related mitochondrial disorders.

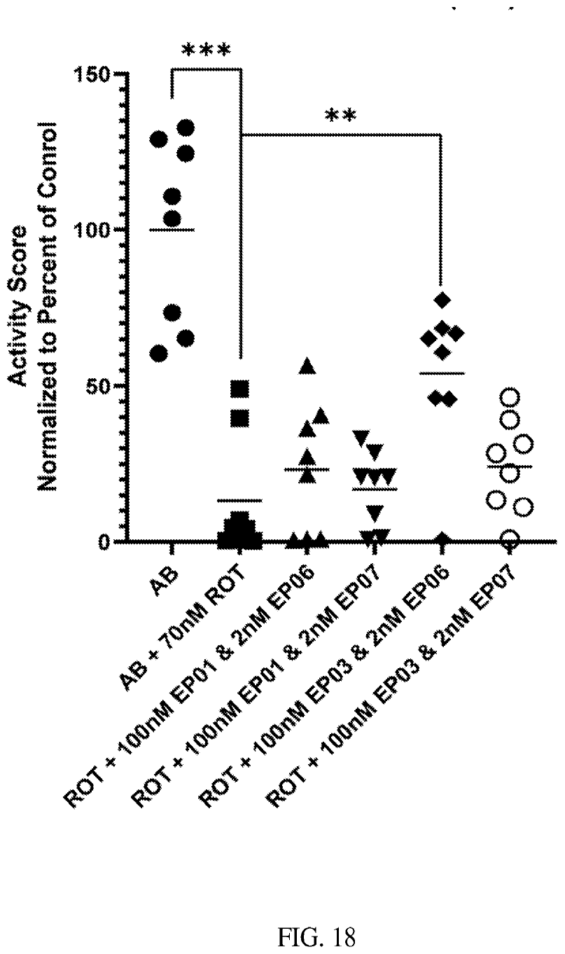

Finally, FIG. 18 demonstrates the potential for synergistic combinations of these flavonoids in different types of mitochondrial disease. Specifically, wild-type (AB) zebrafish exposed on 7 days post fertilization to acute mitochondrial inhibition with high-dose (70 uMol) rotenone had markedly reduced neuromuscular function as evidenced by impaired swimming activity, which was synergistically improved when the wild-type animals were pre-treated for 48 h with 100 nMol (−) epicatechin together with 2 nMol 11-β-hydroxypregnenolone. Thus, flavonoids hold therapeutic potential to prevent neuromuscular decompensation by enhancing cellular resilience to sustain a range of exposures or conditions that acutely impair mitochondrial respiratory chain function.

### CONCLUSIONS

In C. elegans, we observed that (+) epicatechin significantly rescued the shortened lifespan of C. elegans gas-1(fc21) RC deficient nematodes, with the greatest effect seen at 10 nanomolar-range concentration. (+) Epicatechin treatment for 24 hours in gas-1(fc21) worms appears on preliminary analysis to rescue their decreased daf-16 (FOXO1) and par-4 (LKB1) expression as well as increased SOD2 (mnSOD) expression. Replicate analyses are underway. Remarkably, nanomolar-range (+) Epicatechin treatment for 24 to 72 hours fully restored these animals' mitochondrial oxidative balance and significantly increased both their mitochondrial content and membrane potential.

In D. rerio (zebrafish) genetic and inhibitor models of complex I disease, the data show that in ndufs2 (−)+low-dose (12 nM) rotenone zebrafish larvae, swimming activity was significantly improved with 100 nM (+) epicatechin. AB+high-dose (70 nM) rotenone zebrafish larvae swimming activity was significantly improved with 6 nM 11-β-Hydroxypregnenolone. In addition, combining 100 nM (−) epicatechin with 2 nM 11-β-Hydroxypregnenolone led to synergistic improvement in swimming activity in AB+high dose rotenone zebrafish larvae, demonstrating the therapeutic potential of combining flavanoids.

In human cells, very low, nanomolar-range (+) epicatechin consistently improved cell viability in galactose media that requires OXPHOS in a variety of cell types and both pharmacologic and genetic models of RC disease. (+) epicatechin at 1 nMol to 20 nMol range significantly increased mitochondrial content in a human mitochondrial RC disease fibroblast line from a patient with genetic FBXL4 disease. This effect was most pronounced in galactose media, as glucose itself functions as a therapy to significantly improve mitochondrial content in this disease.

In mice, (+) epicatechin improved mitochondrial biogenesis at the level of VDAC expression as well as citrate synthase activity when fed in the drinking water to already sick Pdss2^{kd/kd} animals with a glomerular disease manifesting as frank albuminuria. (+) epicatechin at 5 to 10 μg/mL concentrations for 2 months also led to normalization of their liver mitochondrial content that was depleted relative to wild-type (B6) controls. However, (+) epicatechin did not correct their NAD+ deficiency or altered NADH:NAD+ redox balance, demonstrating the synergistic potential of combining (+) epicatechin with NAD+ agonist therapies such as nicotinic acid, niacin, or niacinamide to correct both mitochondrial depletion (by epicatechin) and their redox imbalance (by NAD+ agonist therapies).

Overall, a “therapeutic cross-training” approach to study evolutionarily-distinct preclinical animal models demonstrated the therapeutic potential of (+)-epicatechin and 11-β-hydroxypregnenolone to ameliorate CI mitochondrial disease survival and neuromuscular phenotypes in two complex I NDUFS24-disease models. In vitro human cell studies and in vivo C. elegans, D. rerio, and M. musculus animal data consistently demonstrated a significant improvement across diverse cellular, mitochondrial, and animal level pathophysiologic parameters that typifies RC disease. No adverse effects of (+) epicatechin were seen in either cell or animal models of primary RC dysfunction. Low (nanomolar range) concentrations of (+) epicatechin were more effective than higher (micromolar range) concentrations in both C. elegans and human cell studies. These data indicate that (+) epicatechin and 11-β-hydroxypregnenolone should significantly improve the health of individuals with mitochondrial disease.

### Example II

### Combination Therapies for Primary RC Disease

Previous work has identified a number of compounds that can be used to advantage in combination with the steroids and flavonoids described in Example I. In previous studies, glucose, N-acetylcysteine, nicotinic acid cysteine bitartrate and probucol have shown promise for the treatment of mitochondrial disease.

As discussed at length above, there are many disorders associated with mitochondrial and RC chain dysfunction. FIGS. 17A, 17B and 17C show that Pdss2^{Kd/Kd} mice relative to untreated mutant controls do not show improvement in symptoms when treated with (+) epicatechin, but that likely epicatechin will be needed to restore mitochondrial content in synergistic combination with an NAD+ agonist therapy to restore the cellular NAD+deficiency that is common in complex I (aka NADH dehydrogenase) disorders and contributes to clinical symptomatology.

In additional studies, the combined effects of (+) epicatechin and 110HP were analyzed. EPM-01 in FIG. 18 is (+)-epicatechin (EMP-01), (−)-epicatechin (EMP-03), 11-hydroxypregnenolone (EPM-06) and 11-hydroxyprogesterone (EPM-07) were assessed in the Zebrafish swimming model described in Example I. Briefly, 3 dpf AB zebrafish larvae (˜ 20 fish per well) were transferred into 6 well plates. The indicated agents were added at 5 and 6 dpf, twice a day. 70 nM rotenone was added on 7 dpf. 4 hours later the larvae were transferred into a 96 well plate (1fish per well, 8 wells per condition), 96-well plates were immediately placed into NOLDUS to evaluate swim activity in repeating light-dark exposure. Swim activity effects were averaged in first 5 minutes of each light-off (‘dark’) period across all light on/off cycles. The results of this assay are shown in FIG. 19. It is clear from this data that (+)-epicatechin and 11-β-hydroxypregnenolone were synergistically protective.

The table provided below lists other agents that may be combined with the flavonoids or steroids described herein to provide efficacious amelioration of mitochondrial disease symptoms.

Water Soluble Drugs
Nicotinic Acid (1 mM)
Riboflavin (10 uM)
Thiamine (25 mM)
L-Carnitine (100 uM)
Folinic Acid (10 uM)
Glucose (10 uM)
DCA (25 mM)
Cysteamine (100 um)
NAC (2.5 Mm)
AICAR (500 uM)
Hydralazine (200 uM)
Lithium Chloride (10 mM)
Cyclohexamide (2 uM)
Arginine (10 mM)
Taurine (800 uM)
Taurine (8 mM)
DMSO Soluble Drugs
Resveratrol (50 uM)
Ethanol Soluble Drugs
Rapamycin (2.5 nM)
Probucol (5 uM)
Lipoic Acid (10 uM)
Vitamin E (250 uM)

### REFERENCES

(1) Zhang Z et al. (2013) Primary respiratory chain disease causes tissue-specific dysregulation of the global transcriptome and nutrient-sensing signaling network. PLOS ONE; 8 (7): e69282.
(2) Zhang Z, Falk M J (2014) Integrated transcriptome analysis across mitochondrial disease etiologies and tissues improves understanding of common cellular adaptations to respiratory chain disease. Int J Biochem Cell Biol; February 22. pii: S1357-2725 (14) 00055-7. doi: 10.1016/j.biocel.2014.02.012.
(3) Tanabe K et al. (2012) Epicatechin limits renal injury by mitochondrial protection in cisplatin nephropathy. Am J Physiol Renal Physiol; 303 (9): F1264-74.
(4) D'Aco K E et al. (2013) Mitochondrial tRNA (Phe) mutation as a cause of end-stage renal disease in childhood. Pediatr Nephrol; 28 (3): 515-19.
(5) Gai X et al. (2013) Mutations in FBXL4, encoding a mitochondrial protein, cause early-onset mitochondrial encephalomyopathy. Am J Hum Genet; 93 (3): 482-95.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A composition for the treatment of mitochondrial disease, comprising an effective amount of at least one agent selected from (+) epicatechin, (−)-epicatechin, 11-β-hydroxypregnenolone, 11-hydroxyprogesterone, probucol, glucose, N-acetylcysteine, cysteine bitartrate, and nicotinic acid, niacin, or niacinamide, in a pharmaceutically acceptable carrier administered separately or in combination.

2. The composition of claim 1, wherein said agent is (+) epicatechin.

3. The composition of claim 1, wherein said agent is 11-β-hydroxypregnenolone.

4. The composition of claim 1, wherein said agent is 11-hydroxyprogesterone,

5. The composition of claim 1, wherein said composition comprises synergistic amounts of (+) epicatechin and 11-β-hydroxypregnenolone.

6. The composition of claim 1, wherein said composition comprises synergistic amounts of (−) epicatechin and 11-β-hydroxypregnenolone.

7. The composition of claim 1, wherein said composition comprises synergistic amounts of (+) epicatechin and 11-hydroxyprogesterone.

8. The composition of claim 1, wherein said composition comprises synergistic amounts of (−) epicatechin and 11-hydroxyprogesterone.

9. The composition of claim 1 further comprising an effective amount of steroids, MAPK-modulators, membrane stabilizers, leukotriene synthesis and receptor inhibitors, inhibitors of IgE isotype switching or IgE synthesis, inhibitors of IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines.

10. A method for alleviating symptoms associated with mitochondrial disease, comprising administration of the composition of claim 1 to a patient in need thereof.

11. The method of claim 10, wherein said symptoms include one or more of muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, dyslipidemia, cardiomyopathy, arrhythmia, anemia, failure to thrive, over or underweight, developmental delay, neurodevelopmental regression, cognitive decline and memory impairment, Parkinsonism, dystonia, liver dysfunction or failure, infertility, metabolic instability, stressor-induced acute decompensation, DLD disease, mitophagy disorders, mitochondrial lipid biogenesis disorders, mitochondrial cofactor disorders, and secondary mitochondrial disorders including but not limited to resulting from toxins, drugs, age, prescribed or illicit medications, smoking, alcohol, environmental exposures, obesity, and genetic disorders that secondarily impair mitochondrial function, structure, or activities.

12. The method of claim 10, wherein said mitochondrial disease is selected from the group consisting of Complex I disease, Complex II disease, Complex III disease, Complex IV disease, Complex V disease, multiple respiratory chain complex disease, adenine nucleotide translocase deficiency, pyruvate dehydrogenase deficiency, mitochondrial depletion disease, multiple mitochondrial DNA deletions disease, mitochondrial DNA maintenance defects, mitochondrial translation defects, mitochondrial nucleotide import disease, Friedreich's ataxia, Leber's Hereditary Optic Neuropathy, Kearns-Sayre Syndrome, Pearson Syndrome, Mitochondrial Myopathy, Mitochondrial Encephalomyopathy with Lactic Acidosis and Stroke-Like Episodes, Myoclonic epilepsy and ragged red fibers, Neurogenic Ataxia and Retinitis Pigmentosa, Mitochondrial Neuro-gastrointestinal encephalomopathy, maternally inherited diabetes and deafness, FBXL4 mitochondrial encephalomyopathy, primary lactic acidosis, Leigh syndrome, Leigh-like syndrome, and multi-system mitochondrial disease.

13. The method of claim 10, wherein said disease in Complex I mitochondrial disease.

14. The method of claim 10, wherein symptoms of mitochondrial disease are alleviated.

15. The method of claim 10, wherein symptoms of acute mitochondrial toxicity are inhibited.

16. The method of claim 10, wherein symptoms of acute decompensation from mitochondrial dysfunction are inhibited.

17. The method for alleviating symptoms associated with mitochondrial disease, comprising administration of the composition of claim 10, said composition comprising (+) epicatechin; or
11-β-hydroxypregnenolone; or
11-hydroxyprogesterone; or
synergistic amounts of (+) epicatechin and 11-β-hydroxypregnenolone; or
synergistic amounts of (−) epicatechin and 11-β-hydroxypregnenolone; or
synergistic amounts of (+) epicatechin and 11-hydroxyprogesterone; or
synergistic amounts of (−) epicatechin and 11-hydroxyprogesterone.

18. The method of claim 17, further comprising administration of effective amounts of one or more agents selected from probucol, glucose, N-acetylcysteine, cysteine bitartrate, and nicotinic acid, niacin, or niacinamide.
